(19) United States
(12) Patent Application Publication
(10) Pub. No.: US 2025/0329410 A1
(43) Pub. Date: Oct. 23, 2025

(54) Systems and Methods for Evaluating Immunological Peptide Sequences

(71) Applicant: The Board of Trustees of the Leland Stanford Junior University, Stanford, CA (US)

(72) Inventors: Maxim Zaslavsky, San Diego, CA (US); Scott D. Boyd, Stanford, CA (US); Anshul Bharat Kundaje, Palo Alto, CA (US); Robert Tibshirani, Palo Alto, CA (US)

(73) Assignee: The Board of Trustees of the Leland Stanford Junior University, Stanford, CA (US)

(21) Appl. No.: 18/709,416

(22) PCT Filed: Nov. 14, 2022
(86) PCT No.: PCT/US2022/079828

§ 371 Date: May 10, 2024

Related U.S. Application Data
(60) Provisional application No. 63/263,912, filed on Nov. 11, 2021.
(60) Provisional application No. 63/362,380, filed on Apr. 1, 2022.

(51) Int. Cl. G16B 15/30; C12Q 1/6806; C12Q 1/6869; G16B 40/20; G16B 40/30; G16B 45/00; G16H 20/10; G16H 50/20; G16H 50/70

(52) U.S. Cl. CPC G16B 15/30; C12Q 1/6806; C12Q 1/6869; G16B 40/20; G16B 40/30; G16B 45/00; G16H 20/10; G16H 50/20; G16H 50/70

(57) ABSTRACT

Systems and methods to assess peptide sequences can incorporate a language model to yield latent representations. Biological properties can be predicted based on latent representations of peptide sequences. Systems and methods to assess immunity status can incorporate one or more models and classifiers to predict health status. Various systems and methods can predict whether an individual is having an active immunological response. Various systems and methods can predict whether an individual is having or has had a particular type of immunological response, such as a pathogenic infection, vaccination, or immunological disorder.

### CROSS REFERENCE TO RELATED APPLICATIONS

This application claims priority to U.S. Provisional Application Ser. No. 63/263,912, entitled “Systems and Methods for Evaluating Immunity,” filed Nov. 11, 2021, and to U.S. Provisional Application Ser. No. 63/362,380, entitled “Systems and Methods for Evaluating Immunological Peptide Sequences,” filed Apr. 1, 2022, each of which is incorporated herein by reference in its entirety.

### STATEMENT REGARDING FEDERALLY SPONSORED RESEARCH OR DEVELOPMENT

This invention was made with Government support under contract DGE1656518 awarded by the National Science Foundation. The Government has certain rights in the invention.

### TECHNOLOGICAL FIELD

The disclosure is generally directed to systems and methods for evaluating, optimizing, and/or generating immunological peptide sequences, including evaluating immunity status and classification of disease status or vaccination status.

### BACKGROUND

B cells and T cells are immunological cells that provide an adaptive immune response to pathogens and vaccines. B cells provide humoral immunity, meaning when matured, B cells produce antibodies to detect pathogens and other foreign bodies for removal. T cells provide cellular immunity, meaning when matured, T cells can detect when a cell of the body is infected or having an abnormal growth of cells and treat the cells in order to remove the infection or growth. To potentiate these responses, B cells and T cells utilize receptors capable of complementing with pathogens such that the pathogen can be detected.

### SUMMARY

Several embodiments are directed to systems and methods for evaluating immunological peptide sequences and/or immunity status. In many embodiments, a predictive classifier or regressor predicts immunity status of an individual, utilizing sequences of B cell receptors and T cell receptors. In several embodiments, a predictive classifier or regressor predicts an individual's prior immunological exposure, utilizing sequences of B cell receptor and T cell receptor. In many embodiments, a predictive model incorporates a language model to extract a latent embedding of immunological peptide sequences or nucleotide sequences encoding immunological peptides. In several embodiments, a trained classifier or regressor is utilized to predict an individual's immunologic or pathogenic disease status, vaccination status, or prior pathogen exposure utilizing the individual's repertoires of B cell receptor and T cell receptor sequences. In some embodiments, a computational system is utilized for linking B cell receptor and T cell receptor sequences with a health status, which can include active immunological activity, active pathogenic infection, recent vaccination, active autoimmune response, an immunodeficiency, prior or active immunological activity of a particular type, prior or active pathogenic infection of a particular pathogen, prior or recent vaccination of a particular vaccine, prior or active autoimmune response of a particular disorder, prior or active immunodeficiency of a particular disorder, a subtype thereof, and/or any combination thereof. In some embodiments, the computational system incorporates a language model to identify similar B cell receptor and T cell receptor sequences. In some embodiments, the computational system includes a language model to evaluate receptor sequence properties, such as complement with a particular antigen, binding specificity, binding affinity, pH binding sensitivity, manufacturability, developability, immunogenicity, or any other sequence-related properties.

### BRIEF DESCRIPTION OF THE DRAWINGS

The description and claims will be more fully understood with reference to the following figures and data graphs, which are presented as exemplary embodiments of the disclosure and should not be construed as a complete recitation of the scope of the disclosure.

FIG. 1 provides a flow diagram of a method to extract embedded representations of peptide sequences using a language model in accordance with various embodiments.

FIG. 2 provides a flow diagram of a method to extract latent embeddings of B cell receptor and T cell receptor peptide sequences using a language model in accordance with various embodiments.

FIG. 3 provides a flow diagram of a method to generate a classifier to detect an active immunological response in accordance with various embodiments.

FIG. 4 provides a flow diagram of a method to cluster B cell receptor and T cell receptor peptide sequences using a language model in accordance with various embodiments.

FIG. 5 provides a flow diagram of a method to assess an individual's health status based on immunological peptide sequences in accordance with various embodiments.

FIG. 6 provides a conceptual illustration of a computational processing system in accordance with various embodiments.

FIGS. 7 and 8 provide a schematic of the framework of Machine Learning for Immunological Diagnosis in accordance with an embodiment.

FIG. 9 provides a data graph depicting the results of fine tuning of the language model in accordance with various embodiments.

FIG. 10 provides a schematic of an ensemble classification pipeline for predicting immune state in accordance with an embodiment.

FIG. 11 provides disease classification performance on held-out test data by the ensemble of three machine learning models of B and T cell repertoires, generated in accordance with various embodiments.

FIG. 12 provides results of ensemble model feature contributions for predicting each class, summarized by whether the features were extracted from BCR or TCR information, generated in accordance with various embodiments.

FIGS. 13A to 13C provide a schematic of the importance of features within a LASSO model (FIG. 13A), a support vector machine model (FIG. 13B), and a random forest model (FIG. 13C), generated in accordance with various embodiments.

FIG. 14 provides a data graph of model prediction confidence for correct versus incorrect predictions, as measured by the difference between the top two predicted class probabilities, generated in accordance with various embodiments. A higher difference implies that the model is more certain in its decision to predict the winning disease label, whereas a low difference suggests that the top two possible predictions were a toss-up.

FIGS. 15A and 15B provides classification prediction performance based on demographic data in a BCR model (FIG. 15A) and a TCR model (FIG. 15B), generated in accordance with various embodiments.

FIG. 16 provides classification performance based on demographic features alone (top panel), demographic features along with sequence features (middle panel), and sequence features only with demographic features regressed out (bottom panel), generated in accordance with various embodiments.

FIGS. 17 to 19 provide disease patient-originating BCR sequences ranked by predicted disease class probability, showing high ranks for IGHV genes known to be disease-associated and for CDR-H3 length patterns reflecting selection for Covid19 (FIG. 17), lupus (FIG. 18), and HIV (FIG. 19), generated in accordance with various embodiments.

FIGS. 20A and 20B provide IGHV gene use proportions in healthy control samples, stratified by ancestry for BCR (FIG. 20A) and TCR (FIG. 20B), generated in accordance with various embodiments. Averages and 95% confidence intervals are shown.

FIGS. 21A to 21C provide disease patient-originating TCR sequences ranked by predicted disease class probability, showing high ranks for TRBV genes known to be disease-associated and for CDRB length patterns reflecting selection for Covid19 (FIG. 21A), lupus (FIG. 21B), and HIV (FIG. 21C), generated in accordance with various embodiments.

FIG. 22 provides a data graph depicting isotype proportions in Covid19, HIV, and lupus patients and healthy individuals, generated in accordance with various embodiments.

FIG. 23 provides data graphs depicting disease patient-originating sequences ranked by predicted disease class probability and grouped by isotype, generated in accordance with various embodiments. Significance was tested for each isotype pair in each panel. **** means p<=1e-4 by two-sided Wilcoxon rank-sum test, with Bonferroni multiple hypothesis testing correction across all tests in all panels.

FIG. 24 provides data graphs depicting: IGHV gene usage in the entire external database of known SARS-COV-2 binding antibody sequences, versus IGHV gene usage in the subset also found in the independent cohorts used to train the disease classification models described here (top panel), and epitope specificities of the entire external database of known SARS-COV-2 binding antibody sequences, versus epitope specificities for the subset also found in the independent cohorts used to train the disease classification models described here (bottom panel), generated in accordance with various embodiments.

FIG. 25 provides data graphs of BCR sequences in the data convergent to known SARS-COV-2 binders, which were ranked significantly higher than other sequences by the model, generated in accordance with various embodiments (one-sided Wilcoxon rank-sum test, U statistic=5.2e8, p value ˜0). Non-overlapping sequences may include additional SARS-COV-2 binders not yet identified in the literature.

FIG. 26 provides a schematic of the cross validation strategy, utilized in accordance with various embodiments.

FIG. 27 provides a data table of kBET batch effect measurements, generated in accordance with various embodiments. Average rejection rate of the null hypothesis that the batch distribution in a sequence's local neighborhood is the same as the global batch distribution (reporting average+/−standard deviation across 3 folds). Closer to 0 indicates the null hypothesis is rarely rejected and suggests the batches are well mixed.

FIGS. 28A and 28B provide IGHV (FIG. 28A) and TRBV (FIG. 28B) gene proportions in each cohort, generated in accordance with various embodiments, the highest proportion each V gene represents of any disease cohort was calculated, and the median of these proportions was plotted (overlaid dashed line). Rare V genes that did not exceed the purple dashed line in at least one disease were then filtered out.

FIGS. 29A and 29B provide stacked bar plots representing how prevalent each IGHV (FIG. 29A) and TRBV (FIG. 29B) gene is by disease, after filtering out rare V genes, generated in accordance with various embodiments.

### DETAILED DESCRIPTION

Turning now to the drawings and data, the various embodiments of systems and methods for evaluating immunological peptide sequences are described. In several embodiments, a language model is utilized to interpret immunological peptide sequence semantics by extracting latent properties from each sequence. In many embodiments, the language model converts immunological peptide sequences into vectors, the vectors having the extracted latent embeddings of the peptide sequence. Various embodiments analyze the peptide sequences via the extracted embeddings. In some embodiments, the extracted embeddings are clustered by similarity, revealing clusters of peptides with similar properties. In some embodiments, a classifier is generated to predict an immunological property based on the extracted embeddings. In some embodiments, a classifier is utilized to predict a function of a particular peptide. For instance, antigen complementation of a particular peptide can be predicted. In some embodiments, a classifier is utilized to make a global prediction of a collection of peptides. For instance, the immune status of an individual can be predicted by sampling a collection of their B cell receptor and/or T cell receptor peptides. In some embodiments, de novo immunological peptide sequences are synthesized that would have a particular biological property.

In accordance with several embodiments, a language model is utilized to interpret immunity status via complementary determining region (CDR) peptide sequences of B cell receptors and/or T cell receptors. In many embodiments, the language model extracts a latent embedding of the B cell receptor and/or T cell receptor sequences. In several embodiments, B cell receptor and/or T cell receptor peptide sequences are derived from cohorts of individuals, each cohort having a particular health status, and a classifier is trained to predict health status utilizing the extracted embeddings of the cohort sequences. In many embodiments, de novo B cell and/or T cell CDR peptide sequences are generated based on latent embeddings having an ability to complement an antigen associated with a particular health status. For example, de novo B cell and T cell CDR peptide sequences can be generated that are complementary to coronavirus, influenza, or other pathogens.

Several embodiments are also directed to generating and training a classifier to detect active immunological activity in an individual (e.g., active pathogenic infection or recent vaccination or acute autoimmune disorder). Accordingly, in many embodiments, peptide sequences of B cell receptors and/or T cell receptors for one baseline cohort and at least one immunologically active cohort are obtained to train the classifier. In some embodiments, the classifier utilizes mutated V gene sequence proportion, V gene counts, and/or J gene counts as features to detect an immunologically active response within an individual. This overall repertoire composition-based classifier may have a variety of classifier outputs. In some embodiments, the prediction task is to detect whether an individual is immunologically active or healthy. In some embodiments, the prediction task is to detect a specific disease or immune disorder type of an individual. In some embodiments, the prediction task is to predict a specific attribute like age, sex, or ancestry.

Many embodiments are directed to generating a classifier to predict health status based on clustering of B cell receptors and/or T cell receptors based on health status. Accordingly, in several embodiments, peptide sequences of B cell and/or T cell receptors for at least two cohorts of individuals, each cohort having a particular health status, are obtained and clustered based on sequence. In many embodiments, the membership of peptide sequences of B cell receptors and/or T cell receptors within clusters associated with a particular health status are utilized to train the classifier.

Several embodiments are directed to utilization of one or more trained computational models to evaluate an individual's immunological status. In many embodiments, a B cell or T cell peptide sequence is utilized within one or more of the trained models to predict one or more of the following immunity statuses: active immunological activity, active pathogenic infection, recent vaccination, active autoimmune response, an immunodeficiency, prior or active immunological activity of a particular type, prior or active pathogenic infection of a particular pathogen, prior or recent vaccination of a particular vaccine, prior or active autoimmune response of a particular disorder, prior or active immunodeficiency of a particular disorder, a subtype thereof, and/or any combination thereof. A subtype can refer to any more specific medical condition, which can be (for example) pathogen subtype, autoimmune disorder subtype, immunodeficiency subtype, vaccine subtype, etc. In many embodiments, an individual's immunity status is evaluated based on their B cell receptor and/or T cell receptor peptide sequences. In several embodiments, a clinical action is performed on the individual based on their immunity status. Clinical actions include (but are not limited to) further clinical evaluation, medicinal treatments, antiviral treatments, antibiotic treatments, autoimmune disorder treatments, vaccination, immunity activation treatments, immunity suppression treatments, diet alterations, and other lifestyle alterations. In several embodiments, an individual is periodically monitored based on their immunity status, and in some embodiments the determination of immunity status is updated routinely during monitoring. In some embodiments, the extracted embeddings provided by the trained language model are projected visually on coordinates, providing a visual aid to monitor immunological activity. In some embodiments, the extracted embeddings from the language model are utilized in a trained classifier to yield classified embeddings that are projected visually on coordinates, which may yield better separation between classes. In some embodiments, the language model and/or the classifier are updated over time to improve visualization of the embeddings. In some embodiments, the visualization of the immunological activity is utilized to perform a clinical action.

Many embodiments are directed to development of antigen complementary peptides, proteins, and/or cells based on B cell or T cell peptide sequence evaluation. In several embodiments, a B cell or a T cell peptide sequence (especially CDR sequences) are evaluated for their ability to provide a particular immunological response, complement with a particular antigen, binding specificity, binding affinity, pH binding sensitivity, manufacturability, developability, immunogenicity, and/or any other property related to receptor sequences. In some embodiments, the B cell or the T cell peptide sequence evaluated is derived from an individual, especially an individual under active and/or recent immunological response. In some embodiments, the B cell or the T cell peptide sequence evaluated is a de novo sequence generated utilizing a language model. Upon evaluation, in accordance with various embodiments, the B cell or the T cell peptide sequence is utilized within an antigen complementary peptide, protein, and/or cell. Antigen complementary peptides and proteins include (but are not limited to) an immunoglobin (Ig), a monoclonal antibody, a nanobody, a B cell receptor, a T cell receptor, a chimeric antigen receptor (CAR), a CDR peptide, and any partial peptide thereof with antigen complementation. Antigen complementary cells include (but are not limited to) a B cell, a T Cell, a CAR T cell, and a hybridoma cell.

Throughout the disclosure is description of computational models to predict or infer an output. It is to be understood that the various computational models can function as classifier or regressor. When the term classifier is utilized to describe the various computational models, it is to be understood that any description of a classifier can also refer to a regressor, unless the output can only be categorical. Likewise, when the term regressor is utilized to describe the various computational models, it is to be understood that any description of a regressor can also refer to a classifier, unless the output can only be numerical. As such, the term classifier or the term regressor should not be limiting to a particular computational function, unless a specific output is described or an alternative output is otherwise impossible.

The term receptor sequence refers to the sequence of immunological receptors, especially B cell receptors and T cell receptors. It is to be understood that a receptor sequence can be a full or partial sequence. Accordingly, a receptor sequence can refer to any of the following: a heavy chain sequence, a light chain sequence, a heavy and light chain sequence, a single CDR sequence, a set of CDR sequences, variable region sequence, constant region sequence, an α chain sequence, a β chain sequence, a γ chain sequence, a δ chain sequence, or any partial sequence thereof. The receptor sequence can also refer to concatenated regions from a full receptor sequence, such as the concatenation of CDR1, CDR2, and CDR3 regions.

### Immunological Peptide Sequence Evaluation

Several embodiments are directed to evaluation of immunological peptide sequences using a language model. In many embodiments, a language model is utilized to extract latent properties of a peptide sequence. Extracted latent embeddings can be utilized to convert peptide sequences into vectors for evaluation. In some embodiments, vectors can be clustered to identify peptides having similar properties and/or functions. In some embodiments, the probability of a particular peptide sequence having a particular property and/or function is determined. In some embodiments, de novo peptide sequences are generated having a predicted property and/or function. In some embodiments, the latent language model is utilized to improve upon itself. To improve upon itself, the language model can change its internal extracted feature to reduce reconstruction error of sequences. In some embodiments, the language model may first be trained on general classes of proteins to learn global rules, then further refined to reduce reconstruction error for immunology-specific sequence patterns. In some embodiments, extracted embeddings are generated from the vectors and utilized to build a classifier to classify sequences as having a particular property and/or function. In some embodiments, extracted embeddings are projected onto coordinates to visualize a collection of sequences (e.g., the repertoire of B cell receptors or the T cell receptors of an individual). In some embodiments, visualization of a collection of sequences allows for quick interpretation of immunological peptide classification and thus quickly determine an overall immunity status for a plurality of immunological conditions, such as (for example) particular immunological activity, particular pathogenic infection, particular autoimmune disorder, particular vaccination status, or particular immunodeficiency disorder.

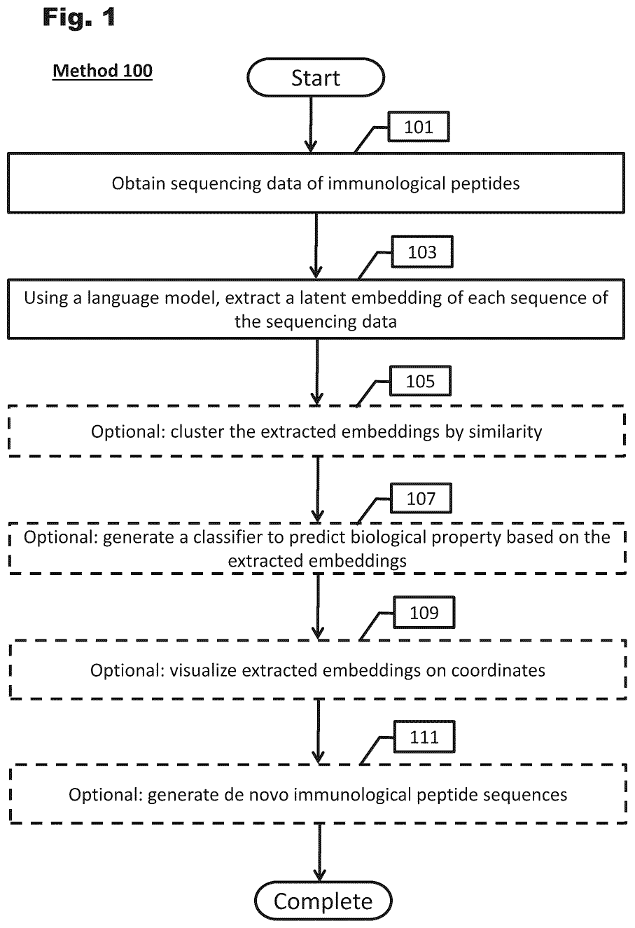

Provided in FIG. 1 is a computational method to extract latent embeddings of immunological peptide sequences using a language model. Method 100 begins with obtaining (101) sequencing data of a collection of immunological peptides. Peptide sequencing data can be obtained by any appropriate method. Generally, nucleic acid molecules and/or proteinaceous species are extracted from biological sample and prepped for sequencing. Any method of sequencing can be utilized. In various embodiments utilizing nucleic acids, high throughput sequencing is performed utilizing a sequencer, such as ones manufactured by Illumina (San Diego, CA). In various embodiments utilizing proteinaceous species, high throughput sequencing is performed utilizing mass spectrometry. Further, a biological sample can be any sample with immunological peptides to be analyzed. Biological samples include (but are not limited to) in vivo samples, in vitro samples, extracted proteinaceous species, isolated proteinaceous species, synthesized proteinaceous species, animal tissue, animal biopsy, bodily fluids (e.g., blood), cell culture, a single cell, healthy samples, and sample biopsies of a medical disorder. In various embodiments, the sequencing data comprises at least 10,000 peptide sequences, 100,000 peptide sequences, at least 1,000,000 peptide sequences, at least 10,000,000 peptide sequences, at least 100,000,000 peptide sequences, at least 1,000,000,000 peptide sequences, at least 10,000,000,000 peptide sequences, at least 100,000,000,000 peptide sequences, or at least 1,000,000,000,000 peptide sequences.

Method 100 extracts (103) a latent embedding of each peptide sequence of the sequencing data utilizing a language model. Any language model capable of extracting latent embeddings can be utilized. Various types of language models can be utilized, such as (for example) neural networks, k-mer embeddings, unigram models, n-gram models, and exponential models. In some embodiments, the language model is a neural network trained to reconstruct protein sequences that have been masked or corrupted. Various architectures of neural networks can be utilized, such as (for example) Long short-term memory (LSTM), transformers, and variational autoencoders. In many embodiments, the language model is capable of extracting a latent embedding of each peptide sequence regardless of its amino acid length.

In several embodiments, the latent language model extracts features and transforms the features into a vector. To achieve its task, in several embodiments, the language model compresses each peptide sequence into an internal, low-dimensional embedding that captures important traits, which are chosen through optimization. Each iteration of model training refines the set of transformations used first to compress a masked sequence, then to restore an unmasked sequence from its low-dimensional version. In many embodiments, the transformation weights that deliver better reconstruction accuracy are accepted. If the final model can successfully un-mask protein sequences, the internal compression and uncompression has extracted fundamental features that summarize the input sequence. Accordingly, in several embodiments, the language model is improved with each sequence utilized for training and/or assessment.

Any peptide sequences can be utilized to train the language model. In some embodiments, a diverse set of proteins from all over the various biological kingdoms are utilized. In some embodiments, proteins of a particular species (e.g., Homo sapiens) are utilized. In some embodiments, a specific class of proteins is utilized. For example, in some embodiments, B cell receptor and/or T cell receptor sequences are utilized, providing an immunological language model. In some embodiments, human B cell receptor and/or T cell receptor sequences are utilized. In some embodiments, the language models are fine-tuned with antibody structural information; for example, the pre-trained language model can be further fine-tuned to reduce error for predicting amino acid contact maps. In some embodiments, a language model is initially trained on general proteins and peptides and then further trained on a particular class of sequences such that the model learns general rules first then more specific rules of the particular class. In some embodiments, training is performed with supervision, which can include reconstruction error and/or knowledge of class labels of the sequences. For example, B cell receptor and T cell receptor sequences with known antigen complementation can be labeled with a particular antigen and/or disease label (e.g., coronavirus and/or COVID19 and/or spike protein; or influenza virus and/or flu and/or haemagglutinin). In some embodiments, a model is trained with a mixture of unsupervised and supervised learning. For example, the language model can be trained in an unsupervised fashion on unlabeled protein sequences from a variety of sources, then is fine-tuned in a supervised manner on labeled immune protein sequences.

Method 100 can optionally cluster (105) the latent embeddings by similarity. By converting the peptide sequences into vectors, the numerical values of the vectors can be utilized to find similar peptide sequences, because the vectors are based on latent embeddings that signify similar properties and/or functions. Furthermore, a peptide sequence can be assessed to determine its cluster membership, providing a prediction of its properties and/or functions. The properties and/or functions can be determined by clusters that contain sequences derived from individuals with the same medical disorder or biological property.

Method 100 also optionally generates (107) a classifier or regressor to predict a biological property and/or function based on its extracted latent embedding. Any type of classifier or regressor can be utilized, such as (for example) logistic regression, LASSO, gradient boosted trees, neural network, nearest neighbors, decision trees, or support vector machine. In various embodiments, peptide sequences having known or suspected properties and/or function can be utilized in the language model to extract their latent embeddings. These latent embeddings can be associated with the known properties and/or function of the peptide sequence. Thus, a classifier can be generated based on the latent embeddings and known properties and/or function.

In some embodiments, the classifier is a separate model and uses the extracted language model embeddings. In these embodiments, the extracted latent embeddings are labeled and used for supervised training. Alternatively, in some embodiments, the classifier is incorporated within the language model and the language model is trained with supervision and labels on the peptide sequences. Whether to incorporate the classifier or keep separate will depend, in part, on whether it is desired to train a language model for a particular classification purpose, or to train a language model to interpret immunological peptides generally and so that the latent embeddings can be utilized in multiple classifier models. In some embodiments, a classifier is evaluated, and based on the evaluation additional data can be collected to improve the classification ability.

Furthermore, an immunological peptide sequence can be utilized in the language model and classifier to predict that sequence's properties and/or function. In some embodiments, a peptide sequence having unknown property and/or function is assessed and classified.

In some embodiments, sequence classifications can be related to sequence properties. For example, sequences can be ranked by their predicted probabilities from a classification model for a particular prediction task. Then the distribution of V gene usage, CDR3 length, isotype usage, sequence motif, peptide properties, amino acid constituency or composition, or amino acid properties can be evaluated versus sequence rank.

Method 100 can also visualize (109) the extracted embeddings on coordinates, which can enable the ability to visualize the various collections of sequences analyzed. For instance, visualization of embeddings can allow for the quick determination of an overall immunity status that allows for facile identification of immunological activities within the collection of sequences. To visualize embeddings, in some embodiments, a UMAP plot or PCA plot is generated. In some embodiments, plots of pairs of embedding dimensions are generated, where each dimension may correspond to a prediction class. In some embodiments, predicted class logit scores are plotted for pairs of classes.

In some embodiments, the collections of sequences to be analyzed are the repertoire of B cell receptor and/or T cell receptor sequences of an individual and visualization of extracted embeddings allows for facile identification of exposure of particular pathogens, any particular autoimmune disorders, any particular immunodeficiency disorders, and/or vaccination status of particular vaccines. In some embodiments, the repertoire of B cell receptor and/or T cell receptor sequences of an individual are assessed over time and visualization of extracted embeddings allows for detection of changes related to exposure of particular pathogens, any particular autoimmune disorders, any particular immunodeficiency disorders, and/or vaccination status of particular vaccines. Changes that can be assessed include (but are not limited to) newly acquired immunological activity, waning immunological activity, and an overall presence or absence of immunology activity, each of which can be assessed globally or for a particular set of one or more medical disorders. Accordingly, various medical disorders can be monitored, including (but not limited to) acquisition of an infection of a particular pathogen, waning immunity to a particular pathogen, severity of an autoimmune disorder, treatment of an autoimmune disorder, severity of an immunodeficiency disorder, treatment of an immunodeficiency disorder, acquisition of neoplastic growth (e.g., cancer), severity of a neoplastic growth, and/or treatment of a neoplastic growth.

Several embodiments are directed to performing a clinical action based on visualization of extracted embeddings on coordinates. Depending on the assessment made by the visualization of extracted embeddings, a clinical action can be performed when immunological activity and/or a change of immunological activity is detected. Clinical actions include (but are not limited to) further clinical evaluation, medicinal treatments, antiviral treatments, antibiotic treatments, autoimmune disorder treatments, vaccination, immunity activation treatments, immunity suppression treatments, diet alterations, and other lifestyle alterations. For instance, upon detection of a medical disorder (such as a pathogenic infection, autoimmune disorder, immunodeficiency disorder, neoplastic growth, etc.), an individual can be further assessed to confirm the status of the medical disorder and/or treated for the medical disorder. In some instances, the severity of a medical disorder and/or success of treatment is monitored over time and based on changes of severity and/or success, modification of a treatment regimen is performed. In some instances, maintenance of immunity to particular antigen is monitored, and in some cases revaccination of a particular pathogen is performed when immunity wanes, or repeat of allergy immunotherapy if tolerance wanes, or repeat of cancer immunotherapy in the case of residual disease, cancer recurrence, or poor response to treatment, or in some cases a treatment for an autoimmune disorder is modified and/or terminated when immunity wanes.

Method 100 can also optionally generate (111) de novo immunological peptide sequences. De novo peptide sequences are sequences generated in silico based on the language model and embeddings. In some embodiments, de novo peptide sequences are generated to have a predicted property and/or function, as can be determined by clustering methods, classification methods, and/or visualization methods. In some embodiments, generated de novo peptide sequences are utilized to synthesize peptides, proteins, receptors, medicinal biologics, or other proteinaceous species. Peptides, proteins, or other proteinaceous species can be chemically synthesized (e.g., solid phase peptide synthesis) or biologically synthesized (e.g., recombinant expression systems).

In one exemplary method to generated de novo sequences, V and J segments are developed and selected that are predicted to have some specific antigen complementation or are otherwise associated with a particular disease. Keeping V and J segments the same, CDR3 sequences are mutated. When generating BCR de novo sequences, CDR1 and CDR2 can be mutated as well. The mutated sequences are scored in silico via a predictive model. In addition, further mutational analysis on scored sequences can be performed in an iterative fashion to find sequences with enhanced binding ability. Furthermore, the predictive model can also incorporate various sequence properties and sequences can be further scored and selected based on these properties. Sequence properties that may be useful include (but are not limited to) complement with a particular antigen, binding specificity, binding affinity, pH binding sensitivity, manufacturability, developability, or immunogenicity. Based on scores and/or desired properties, sequences can be selected for synthesis of proteinaceous species (e.g., synthesis of peptide, receptor, medicinal biologics, etc.).

While specific examples of processes for extracting latent embeddings of peptide sequences utilizing a language model are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for extracting latent embeddings of peptide sequences utilizing a language model appropriate to the requirements of a given application can be utilized in accordance with various embodiments of the invention.

### Immunity Evaluation

Several embodiments are directed to evaluation of B cell receptor and/or T cell receptor sequences using one or more models to evaluate immunity. In many embodiments, sequences of a B cell receptor and/or of T cell receptor are utilized to evaluate immunity. In some embodiments, CDR1 sequence, CDR2 sequence, CDR3 sequence, V gene segment selection, or any combination thereof of a B cell receptor and/or of T cell receptor is utilized to evaluate immunity. Also the HLA type of the individual can be used for T cell receptor evaluation. Various computational models can be utilized to analyze B cell receptor and/or T cell receptor sequences to evaluate immunity, including (but not limited to) a protein sequence language model, a classifier to predict immunity status based on extracted latent embeddings extracted by a language model, a classifier to predict an active immune response, a clustering model to cluster peptides based on sequence similarity, and a classifier to evaluate immunity status-based peptide sequence cluster membership.

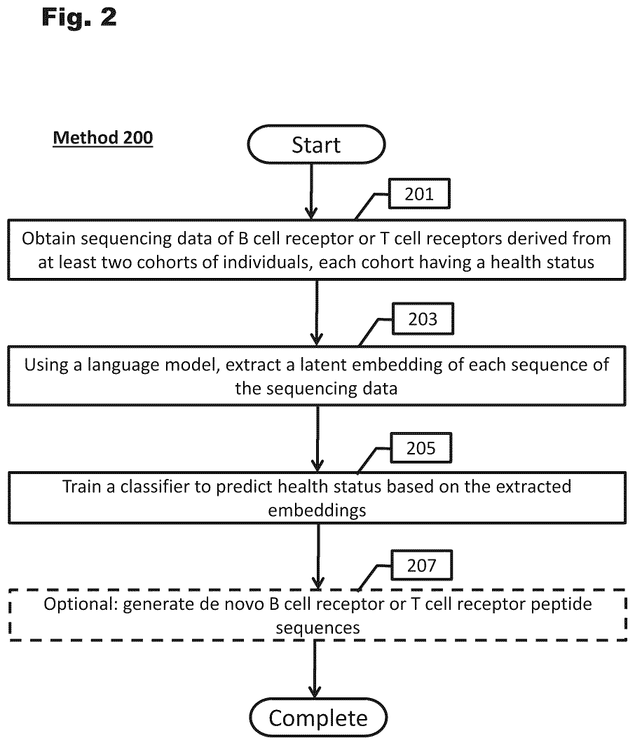

Several embodiments are directed to utilizing a language model and a classifier to assess B cell receptor and/or T cell receptor sequences for determining particular immunological responses as part of an immunity status. Provided in FIG. 2 is a computational method to extract latent embeddings of B cell receptor and/or T cell receptor sequences and utilize a classifier to predict a health status. Method 200 obtains (201) sequencing data of B cell receptors and/or T cell receptors derived from at least two cohorts of individuals, each cohort having a health status. In various embodiments, the sequencing data comprises at least 100,000 unique receptor sequences per individual, at least 1,000,000 unique receptor sequences per individual, at least 10,000,000 unique receptor sequences per individual, at least 100,000,000 unique receptor sequences per individual, at least 1,000,000,000 unique receptor sequences per individual, at least 10,000,000,000 unique receptor sequences per individual, at least 100,000,000,000 unique receptor sequences per individual, or at least 1,000,000,000,000 unique receptor sequences per individual. In various embodiments, the sequencing data comprises at least 10 people per cohort, at least 100 people per cohort, at least 1000 people per cohort, or at least 10,000 people per cohort.

The health status can be any status related to B cell or T cell immunity, including (but not limited to) healthy, active immunologic response, and prior immunologic response. A healthy status refers to an individual that can be utilized as baseline comparison, meaning the individual has not been affected by a particular active or prior immunological response. An active immunological response refers to an individual having a particular immunological response resulting in active B cell or T cell generation. Active immunological responses include (but are not limited to) an active pathogenic infection, an autoimmune disorder, an active acute autoimmune reaction, a recent vaccination, multiples thereof (e.g., two active pathogenic infections), and any combination thereof (e.g., active pathogenic infection and active vaccination). A prior immunological response refers to an individual having an immunological response resulting in B cell or T cell generation, but is no longer actively generating or stimulating B cells or T cells, though quiescent memory B cells or T cells may be circulating. Prior immunological responses include (but are not limited to) a prior pathogenic infection, a prior vaccination, multiples thereof (e.g., two prior pathogenic infections), and any combination thereof (e.g., prior pathogenic infection and prior vaccination). In some embodiments, a cohort is defined by having a particular immunological response, such as (for example) an active SARS-COV2 infection, a prior SARS-COV2 infection, a recent COVID19 vaccination, a prior COVID19 vaccination, an active systemic lupus erythematosus (SLE) disorder, and an acute SLE flare. While only a few particular immunological responses are offered as examples, it is to be understood that a cohort can be defined by any particular immunological response or a combination of two or more immune responses.

The sequencing data should include peptide sequences of B cell receptors and/or T cell receptors, especially CDR regions. To generate peptide sequences, in accordance with some embodiments, genetic material (e.g., DNA or RNA) is extracted from B cells and/or T cells and sequenced utilizing a nucleic acid sequencer and peptide sequences are inferred from the nucleic acid sequencing results.

Method 200 utilizes a language model to extract (203) a latent embedding of each receptor sequence of the sequencing data. Any language model capable of extracting latent embeddings can be utilized. Various types of language models can be utilized, such as (for example) neural networks, k-mer embeddings, unigram models, n-gram models, and exponential models. In some embodiments, the language model is a neural network trained to reconstruct protein sequences that have been masked or corrupted. Various architectures of neural networks can be utilized, such as (for example) Long short-term memory (LSTM), transformers, and variational autoencoders. In many embodiments, the language model is capable of extracting a latent embedding of each peptide sequence regardless of its amino acid length.

B cell receptor and T cell receptor sequences can be utilized to train the language model, providing an immunological language model. In some embodiments, human B cell receptor and/or T cell receptor sequences are utilized.

In several embodiments, the latent language model extracts features and transforms the features into a vector. To achieve its task, in several embodiments, the language model compresses each peptide sequence into an internal, low-dimensional embedding that captures important traits, which are chosen through optimization. Each iteration of model training refines the set of transformations used first to compress a masked sequence, then to restore an unmasked sequence from its low-dimensional version. In many embodiments, the transformation weights that deliver better reconstruction accuracy are accepted. If the final model can successfully un-mask protein sequences, the internal compression and uncompression has extracted fundamental features that summarize the input sequence. Accordingly, in several embodiments, the language model is improved with each sequence utilized for training and/or assessment.

In many embodiments, the extracted latent embedding of each sequence is converted into a numerical vector, which can be clustered to identify sequence vectors having similar antigen complementation. By comparing clusters of at least two cohorts, particular clusters and peptide sequence members within those cohorts can be identified as having antigen complementation resulting from a particular health status associated with the cohort.

Method 200 can utilize the extracted latent embeddings associated with a particular health status to train (205) a classifier or regressor model to predict health status. Any type of classifier or regressor can be utilized, such as (for example) logistic regression, LASSO, gradient boosted trees, neural network, nearest neighbors, decision trees, or SVM. A classifier can be incorporated into the language model or can be a separate from the language model. When incorporated into the language model, the classifier can be trained with supervision by labeling the input sequences and the classification can be performed concurrently with the extraction of embeddings. When a classifier is separate from the language model, the classifier can be trained with supervision by labelling the extracted embeddings and utilizing the embeddings as input. It should be understood that the classifier model can be trained with a plurality of sets of extracted latent embeddings, each set associated with a particular health status. The number of sets of extracted latent embeddings is limitless, and thus a classifier can predict the health status of an infinite number of health statuses. Accordingly, in various embodiments, at least two sets of extracted latent embeddings, at least three sets of extracted latent embeddings, at least four sets of extracted latent embeddings, at least five sets of extracted latent embeddings, at least six sets of extracted latent embeddings, at least seven sets of extracted latent embeddings, at least eight sets of extracted latent embeddings, at least nine sets of extracted latent embeddings, at least ten sets of extracted latent embeddings, or greater than ten sets of extracted latent embeddings are utilized to train the classifier, wherein each set is derived from a cohort of individuals associated with a unique disease status.

The parameters of a trained classifier can be optimized and/or fine-tuned. In some embodiments, the immunodeficiency and/or specificity of a classifier can be modified to fit the needs of the classification to be performed. For instance, immunodeficiency and/or specificity thresholds may be modified based on immunological seasons (e.g., influenza season), changes in viral subtype (e.g., coronavirus variant changes), or baseline infection levels. In some embodiments, a classifier utilizes abstention to abstain from classifying a B cell receptor sequence or a T cell receptor sequence, or from classifying an individual as having a particular immunity status.

In some embodiments, the training or evaluation sequences can be filtered down to sequences likely to correspond to the disease class. For example, an unsupervised nearest neighbors graph can be constructed from sequence embedding vectors, where each sequence is one node connected to several nearby sequences. Certain sequences can be excluded from the training set, such as if their graph neighborhoods include sequences from individuals of many immune states (which can indicate these sequences are common background sequences and not actually related to a particular immune state) or if their graph neighborhoods only have sequences from a minority of individuals of the same cohort (which can indicate rare sequences not shared across individuals). Classification performance may improve by training the classifier on meaningful sequences, or on all sequences but with certain sequences assigned higher sample weight. For an evaluation set sequence, its nearest neighbors in the training set may also be evaluated by similar heuristics; some evaluation set sequences may not be meaningful to include in overall repertoire classification.

The trained classifier can be utilized to assess a B cell receptor sequence or a T cell receptor sequence to determine the association of the sequence with some classification (e.g., association with a particular medical disorder or disease). Furthermore, the classifier can be utilized to assess the repertoire of B cell receptors and/or T cell receptors of an individual to determine whether the individual has a particular health status. In some embodiments, classification predictions for an entire patient sample repertoire, or other collection of sequences, are created by aggregating individual sequence predictions. In some embodiments, individual sequence predictions may be aggregated with a trimmed mean operation to produce a central estimate of sequence classifications robust to the background or noisy sequences in a repertoire or other collection of sequences. In some embodiments, sequence predictions are aggregated by sequence confidence weights. In some embodiments, sequence predictions are aggregated by a combination of approaches, such as a weighted trimmed mean or weighted and/or trimmed median that incorporates sequence confidence weights derived from nearest-neighbors graph connectivities or other methods. In some embodiments, a classifier is evaluated, and based on the evaluation additional data can be collected to improve the classification ability.

When classifying a person-level or sample-level status, different collections of immune receptors may be used depending on the prediction task. In some embodiments, somatic hypermutation frequencies in non-class switched (IgD/IgM) or class-switched (IgA/IgG/IgE) B cell receptors are used for prediction of disease, health status, age, sex, ancestry, medication history or environmental exposures.

In some embodiments, sequences from the cohort that are identified to associate with the classification are selected to be synthesized. In various embodiments, a score generated by the classifier or regressor is utilized to select sequences having desired association, such as association with a particular disorder or complementation with an antigen. In some embodiments, the classifier is further trained with sequences having known properties, such as complement with a particular antigen, binding specificity, binding affinity, pH binding sensitivity, manufacturability, developability, immunogenicity, or any other sequence-related properties. And thus, in some embodiments, a sequence is selected based on one or more sequence properties. In some embodiments, selected peptide sequences are utilized to synthesize antigen complementary proteinaceous species, which can be chemically synthesized (e.g., solid phase peptide synthesis) or biologically synthesized (e.g., recombinant expression systems). Peptides, proteins, receptors, medicinal biologics, or other proteinaceous species can be synthesized.

Method 200 can also optionally generate (207) de novo B cell receptor or T cell receptor peptide sequences. De novo peptide sequences are sequences generated in silico based on the language model and latent embeddings. In some embodiments, de novo peptide sequences are generated to have a predicted antigen complementation, as can be determined by clustering methods and/or classification methods. In some embodiments, de novo peptide sequences are utilized to synthesize antigen complementary proteinaceous species, which can be chemically synthesized (e.g., solid phase peptide synthesis) or biologically synthesized (e.g., recombinant expression systems). Peptides, proteins, receptors, medicinal biologics, or other proteinaceous species can be synthesized.

While specific examples of processes for predicting health status based on extracted latent embeddings are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for predicting health status based on extracted latent embeddings appropriate to the requirements of a given application can be utilized in accordance with various embodiments of the invention.

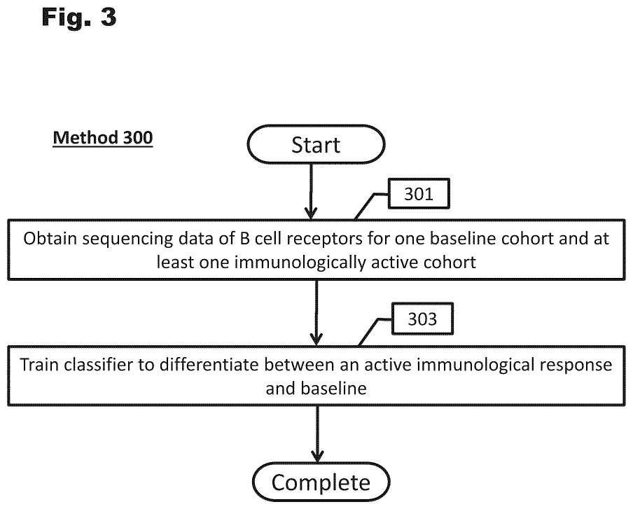

Several embodiments are directed to utilizing a computational model to determine whether an individual has an active immune response as part of determining an overall immunity status. Provided in FIG. 3 is a method to generate a classifier to detect the hallmarks of an immunological response, including whether there is an active immunological response, the disorder, infection, or vaccination related to the immunological response, and/or traits of the individual assessed (e.g., age group). Method 300 obtains (301) sequencing data of B cell receptors derived from at least one baseline cohort and at least one immunologically active cohort. In various embodiments, the sequencing data comprises at least 100,000 unique receptor sequences per individual, at least 1,000,000 unique receptor sequences per individual, at least 10,000,000 unique receptor sequences per individual, at least 100,000,000 unique receptor sequences per individual, at least 1,000,000,000 unique receptor sequences per individual, at least 10,000,000,000 unique receptor sequences per individual, at least 100,000,000,000 unique receptor sequences per individual, or at least 1,000,000,000,000 unique receptor sequences per individual. In various embodiments, the sequencing data comprises at least 10 people per cohort, at least 100 people per cohort, at least 1000 people per cohort, or at least 10,000 people per cohort.

At least one immunologically active cohort can be a collection of individuals having an active immune response, especially an acute immune response that results in B cell stimulation in maturity. Active immunological responses include (but are not limited to) an active pathogenic infection, an autoimmune disorder, an active acute autoimmune reaction, an immune dysfunction, a recent vaccination, multiples thereof (e.g., two active pathogenic infections), and any combination thereof (e.g., active pathogenic infection and active vaccination). In some embodiments, a cohort is defined by having a particular immunological response, such as (for example) an active SARS-COV2 infection, a recent COVID19 vaccination, a prior COVID19 vaccination, and an acute SLE flare. A baseline cohort is a collection of individuals that are not currently undergoing an active immune response, such that a baseline immune response can be established.

Any hallmark of an active immunological response detectable via sequencing can be assessed to differentiate between an active response and a baseline response. For instance, when naïve B cells are activated, the B cells switch into the IgG and IgA isotypes. In some embodiments, the ratio of IgG or IgA isotypes is compared to the total IgG to detect an active response. In some embodiments, the ratio of IgG or IgA isotypes is compared to IgM and/or IgD isotypes. In some embodiments, the rate of somatic hypermutation is utilized to assess active immune response. In some embodiments, the proportion of sequences that are hypermutated is utilized to assess active immune response. In some embodiments, a count of V genes and/or count of J genes is utilized to assess active immune response.

Method 300 also trains (303) a classifier or regressor to differentiate between an active immune response and baseline immune response. Any type of classifier or regressor can be utilized, such as (for example) logistic regression, LASSO, gradient boosted trees, neural network, nearest neighbors, decision trees, or SVM. In some embodiments, the classifier is a binary linear model with elastic net regularization. In several embodiments, the classifier is trained by associating one or more hallmarks of an active immune response that is differentiated between the cohort having the active immune response and the baseline cohort. In some embodiments, the classifier is trained to detect an active immune response of a particular type (e.g., coronavirus infection). In some embodiments, a classifier is evaluated, and based on the evaluation additional data can be collected to improve the classification ability. In some embodiments, individual sequence predictions by a classifier may be aggregated with a trimmed mean operation to produce a central estimate of sequence classifications robust to the background or noisy sequences in a repertoire or other collection of sequences; thus a sequence-level classifier can become a patient-level or sample-level classifier.

The parameters of a trained classifier can be optimized and/or fine-tuned. In some embodiments, the sensitivity and/or specificity of a classifier can be modified to fit the needs of the classification to be performed. For instance, sensitivity and/or specificity thresholds may be modified based on immunological seasons (e.g., influenza season), changes in viral subtype (e.g., coronavirus variant changes), or baseline infection levels. In some embodiments, a classifier utilizes abstention to abstain from classifying an individual as having an active immune response or baseline response.

Furthermore, several embodiments are directed to utilizing the classifier to determine whether an individual is having an active immune response. Accordingly, an individual can have their B cell receptors and/or T cell receptors sequenced and the sequencing data entered within a trained classifier to detect one or more hallmarks associated with an active immune response. In various embodiments, the individual's sequencing data comprises at least 100,000 unique receptor sequences, at least 1,000,000 unique receptor sequences, at least 10,000,000 unique receptor sequences, at least 100,000,000 unique receptor sequences, at least 1,000,000,000 unique receptor sequences, at least 10,000,000,000 unique receptor sequences, at least 100,000,000,000 unique receptor sequences, or at least 1,000,000,000,000 unique receptor sequences.

While specific examples of processes for training a classifier to detect an active immunological response are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for training a classifier to detect an active immunological response appropriate to the requirements of a given application can be utilized in accordance with various embodiments of the invention.

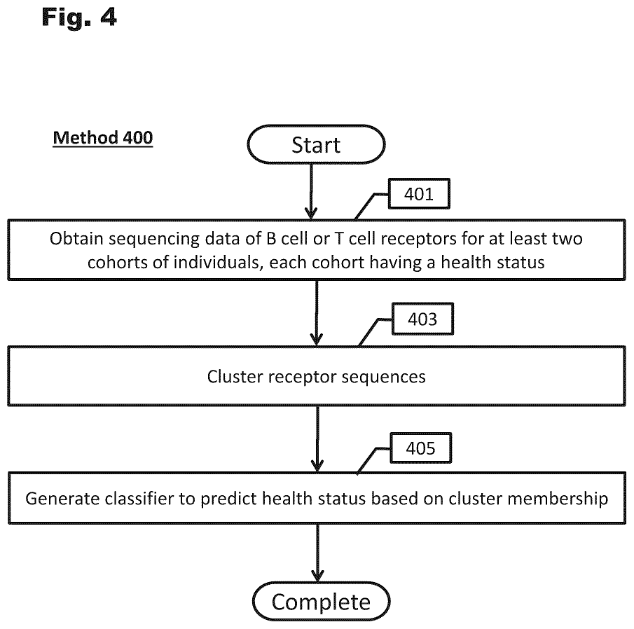

Several embodiments are directed to clustering B cell receptor and/or T cell receptor sequences based on similarity to determine whether a particular receptor sequence is associated with a particular immunological response as part of evaluating an immunity status. Provided in FIG. 4 is a method to cluster B cell receptor and/or T cell receptor sequences and utilize a classifier to predict a health status. Method 400 obtains (401) sequencing data of B cell receptors or T cell receptors derived from at least two cohorts of individuals, each cohort having a health status. In various embodiments, the sequencing data comprises at least 100,000 unique receptor sequences per individual, at least 1,000,000 unique receptor sequences per individual, at least 10,000,000 unique receptor sequences per individual, at least 100,000,000 unique receptor sequences per individual, at least 1,000,000,000 unique receptor sequences per individual, at least 10,000,000,000 unique receptor sequences per individual, at least 100,000,000,000 unique receptor sequences per individual, or at least 1,000,000,000,000 unique receptor sequences per individual. In various embodiments, the sequencing data comprises at least 10 people per cohort, at least 100 people per cohort, at least 1000 people per cohort, or at least 10,000 people per cohort.

The health status can be any status related to B cell or T cell immunity, including (but not limited to) healthy, active immunologic response, and prior immunologic response. A healthy status refers to an individual that can be utilized as baseline comparison, meaning the individual has not been affected by a disease state associated with particular active or prior immunological responses. An active immunological response refers to an individual having an immunological response resulting in active B cell or T cell generation or stimulation. Active immunological responses include (but are not limited to) an active pathogenic infection, an autoimmune disorder, an active acute autoimmune reaction, a recent vaccination, multiples thereof (e.g., two active pathogenic infections), and any combination thereof (e.g., active pathogenic infection and active vaccination). A prior immunological response refers to an individual having a disease state associated with prior immunological responses resulting in B cell or T cell generation, but no longer actively generating or stimulating B cells or T cells. Prior immunological responses include (but are not limited to) a prior pathogenic infection a prior vaccination, multiples thereof (e.g., two prior pathogenic infections), and any combination thereof (e.g., prior pathogenic infection and prior vaccination). In some embodiments, a cohort is defined by having a particular immunological response, such as (for example) an active SARS-COV2 infection, a prior SARS-COV2 infection, a recent COVID19 vaccination, a prior COVID19 vaccination, an active systemic lupus erythematosus (SLE) disorder, and an acute SLE flare.

The sequencing data should include peptide sequences, of B cell receptors and/or T cell receptors or at least one of the peptide chain types that comprise BCRs and TCRs. In some embodiments, sequences of CDR3 are utilized for clustering. To generate peptide sequences, in accordance with some embodiments, genetic material (e.g., DNA or RNA) is extracted from B cells and/or T cells and sequenced utilizing a nucleic acid sequencer and peptide sequences are determined from the nucleic acid sequencing results.

Method 400 utilizes a clustering method to cluster (403) the receptor sequences based on sequence similarity. Any clustering method capable of clustering sequences based on similarity can be utilized. Examples of clustering methods include (but are not limited to) k-means clustering, hierarchical clustering, single-linkage clustering, and Louvain community detection. In several embodiments, sequences are clustered by edit distance. In some embodiments, all sequences in cluster share common features, such as (for example) same V gene, same J gene, same sequence length, and sharing a certain percentage of identity (e.g., 85% sequence identity with the cluster's centroid). In some embodiments, clusters are associated with a particular disease when originating from multiple individuals that have or have had the disease. In some embodiments, clusters are discarded if not meeting parameters of disease association, such as (for example) if the sequences are derived from a small number of individuals (e.g., fewer than 3) or if the percentage individuals providing sequences of the cluster is below a threshold (e.g., less than 80% of individuals providing sequences had the disease).

Method 400 can utilize the cluster memberships associated with a particular health status to train (405) a classifier or regressor model to predict health status. Any type of classifier or regressor can be utilized, such as (for example) logistic regression, LASSO, gradient boosted trees, neural network, nearest neighbors, decision trees, or SVM. The trained classifier can be utilized to assess B cell receptor and T cell receptor sequences of an individual to determine whether the individual has a particular health status. In some embodiments, a classifier is evaluated, and based on the evaluation additional data can be collected to improve the classification ability.

The parameters of a trained classifier can be optimized and/or fine-tuned. In some embodiments, the sensitivity and/or specificity of a classifier can be modified to fit the needs of the classification to be performed. For instance, sensitivity and/or specificity thresholds may be modified based on immunological seasons (e.g., influenza season) or baseline infection levels. In some embodiments, a classifier utilizes abstention to abstain from classifying a B cell receptor sequence or a T cell receptor sequence, or from classifying an individual as having a particular immunity status.

Furthermore, several embodiments are directed to utilizing the classifier to predict health status of an individual. Accordingly, in many embodiments, cluster memberships derived from sequencing data of an individual's B cell receptor and T cell receptor sequences are entered into the classifier to predict the individual's health status. In various embodiments, the sequencing data of the individual comprises at least 100,000 unique receptor sequences, at least 1,000,000 unique receptor sequences, at least 10,000,000 unique receptor sequences, at least 100,000,000 unique receptor sequences, at least 1,000,000,000 unique receptor sequences, at least 10,000,000,000 unique receptor sequences, at least 100,000,000,000 unique receptor sequences, or at least 1,000,000,000,000 unique receptor sequences.

While specific examples of processes for training a classifier to predict health status based on cluster membership are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for training a classifier to predict health status based on cluster membership appropriate to the requirements of a given application can be utilized in accordance with various embodiments of the invention.

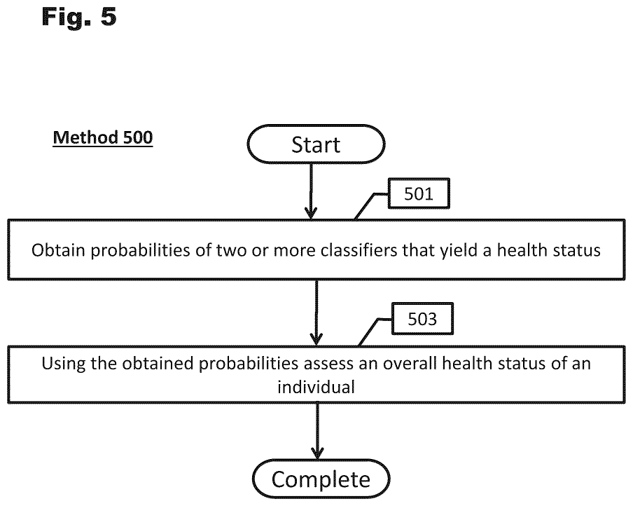

Several embodiments are directed to combining one or more models and classifiers to produce an ensemble model, or a single model trained on the combination of all feature representations, to provide a more encompassing assessment of health status. In various embodiments, one or more methods of Method 200, Method 300, and Method 400 can be combined to yield an ensemble model. Provided in FIG. 5 is a method to utilize each model's predicted probability for each possible class to assess an overall health status of an individual. Method 500 can begin by obtaining (501) the probabilities of two more classifiers that yield a health status. In many embodiments, the two or more classifiers can include at least one of the classifiers described in association with FIGS. 2, 3, and 4. In some embodiments, demographic or biological variables with potential confounding effects, like sex, age, or ancestry, can be regressed out of the input data to the ensemble model.

Using the obtained probabilities, Method 500 assesses (503) a health status of an individual. The obtained probabilities can be utilized as vectors in a classifier or regressor to provide a combined predicted probability vector, yielding an overall health status. Any type of classifier or regressor can be utilized, including (but not limited to) logistic regression, LASSO, gradient boosted trees, neural network, nearest neighbors, decision trees, or SVM. In some embodiments, a multiclass linear SVM is utilized to map the combined the combined predicted probability vectors.

The parameters of a combinatorial classifier can be optimized and/or fine-tuned. In some embodiments, the sensitivity and/or specificity of a classifier can be modified to fit the needs of the classification combination. For instance, sensitivity and/or specificity thresholds may be modified based on immunological seasons (e.g., influenza season), changes in viral subtype (e.g., coronavirus variant changes), or baseline infection levels. In some embodiments, a combinatorial classifier maintains abstention from a classifier utilized to provide the input probabilities.

While specific examples of processes for assessing an overall health status based on combining probabilities of two or more classifiers are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for assessing an overall health status based on combining probabilities of two or more classifiers appropriate to the requirements of a given application can be utilized in accordance with various embodiments of the invention.

### Computational Processing System

A computational processing system to evaluate immunity in accordance with various embodiments of the disclosure typically utilizes a processing system including one or more of a CPU, GPU and/or other processing engine. In some embodiments, the computational processing system is housed within a computing device. In certain embodiments, the computational processing system is implemented as a software application on a computing device such as (but not limited to) mobile phone, a tablet computer, and/or portable computer.

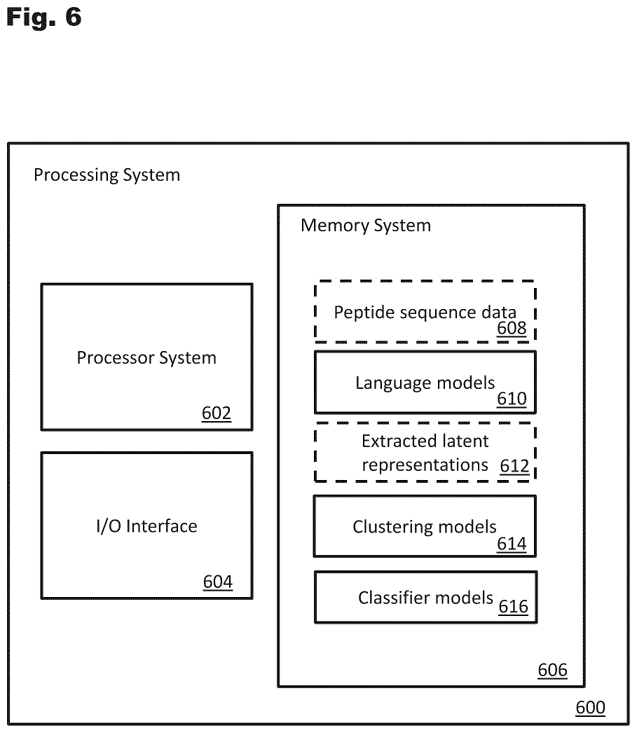

A computational processing system in accordance with various embodiments of the disclosure is illustrated in FIG. 6. The computational processing system 600 includes a processor system 602, an I/O interface 604, and a memory system 606. As can readily be appreciated, the processor system 602, I/O interface 604, and memory system 606 can be implemented using any of a variety of components appropriate to the requirements of specific applications including (but not limited to) CPUs, GPUs, ISPs, DSPs, wireless modems (e.g., WiFi, Bluetooth modems), serial interfaces, depth sensors, IMUs, pressure sensors, ultrasonic sensors, volatile memory (e.g., DRAM) and/or non-volatile memory (e.g., SRAM, and/or NAND Flash). In the illustrated embodiment, the memory system is capable of storing language models 610, clustering models 614, and classifier models 616. The various model applications can be downloaded and/or stored in non-volatile memory. When executed the various model applications are each capable of configuring the processing system to implement computational processes including (but not limited to) the computational processes described above and/or combinations and/or modified versions of the computational processes described above. In several embodiments, the language models 610, the clustering models 614, and the classifier models 616 can utilize peptide sequence data 608, which can optionally be stored in the memory system, to perform the various tasks of the models. In certain embodiments, the language model applications 610 can generate extracted latent embeddings 612, which can be optionally stored in memory or utilized without storage. The extracted latent embeddings 612 can be utilized within the clustering models 614 and/or classifier models 616 to evaluate immunity.

While specific computational processing systems are described above with reference to FIG. 6, it should be readily appreciated that computational processes and/or other processes utilized in the provision of immunity evaluation in accordance with various embodiments of the disclosure can be implemented on any of a variety of processing devices including combinations of processing devices. Accordingly, computational devices in accordance with embodiments of the disclosure should be understood as not limited to specific computational processing systems. Computational devices can be implemented using any of the combinations of systems described herein and/or modified versions of the systems described herein to perform the processes, combinations of processes, and/or modified versions of the processes described herein.

### EXEMPLARY EMBODIMENTS

The embodiments of the disclosure will be better understood with the various examples provided within. Provided within is a manuscript and supplements to provide examples of performing the various embodiments as described.

Disease Diagnostics using Machine Learning of Immune Receptors

Modern medical diagnosis relies heavily on laboratory testing for cellular or molecular abnormalities in specimens from a patient, or the presence of pathogenic microorganisms. For autoimmune disorders like lupus or multiple sclerosis, diagnosis via a combination of clinical or imaging observations, detection of autoantibodies, and exclusion of other conditions is a lengthy process that can delay treatment. Evolution has provided vertebrate animals with immune systems that carry out molecular surveillance for abnormal exposures, using B cells and T cells expressing diverse, randomly generated antigen receptors. In response to viruses, vaccines, and other exposures, the repertoire of B and T cell receptors changes in composition, due to clonal expansion of stimulated cells, introduction of additional somatic mutations into B cell receptor genes, and selection processes that further reshape the immune cell populations. In disregulated immunity, self-reactive lymphocytes can also clonally proliferate and cause immunological pathologies.

Being able to interpret the specificities encoded in a patient's adaptive immune system could allow assessment for many infectious diseases at once, as well as offering insight into autoimmune reactions. Tracking immune receptor repertoires has already proved useful in diagnosing lymphocyte malignancies and monitoring cancer treatment responses. But immune repertoire sequencing has largely not been put to use clinically to diagnose, prognosticate, or monitor infectious and autoimmune disease. At issue is the high variability of immune receptor genes due to somatic rearrangement. To overcome this challenge, it was hypothesized that a combination of machine learning techniques for B and T cell sequencing data-including clonal analysis and language modeling-could identify distinct and systematic patterns of disease across people.

Using systematically collected datasets of B cell receptor (BCR) heavy chain (IgH) and T cell receptor (TCR) beta chain (TRB) sequences from peripheral blood, the presence of infectious and immunological diseases was identify by developing and combining three machine learning representations of immune repertoires (FIG. 7). Many investigations of how disease reshapes immune repertoires has relied on identification of nearly identical “convergent” receptor sequences across people with the same disease. In addition, individuals were grouped by inferring broader functional similarities in their immune receptors. Other shared characteristics of immune responses were also detected: the extent of class switching of antibody constant regions, degree of somatic mutation diversification of BCR repertoires, and effects of selection in distorting quantitative features like IgH or TRB complementarity determining region 3 (CDR3) lengths. B and T cell signals were combine for a more complete view of immunity than many earlier analyses limited to either the BCR or TCR repertoire only.

The machine learning process distinguishes healthy from diseased individuals, viral infections from autoimmune or immunodeficiency conditions, and different pathogen infections from each other-without prior knowledge of pathogenesis. This approach also generates interpretable rankings for disease-specific sequences, revealing that the classifier recapitulates independently discovered biological facts, including identifying SARS-COV-2-specific antibodies and T cells.

### Integrated Repertoire Models of Disease States

Even in patients with an active infectious disease, only a fraction of immune receptors may be devoted to the responsible pathogen. To determine an individual's immune status from BCR or TCR sequencing, a diagnostic algorithm must sift through hundreds of thousands of unique sequences to identify the rare specific ones. Candidate disease-specific receptor sequences can be highly variable across individuals. T cell receptor sequences are restricted by an individual's HLA alleles, and B cell receptors show additional sequence diversity due to somatic hypermutation during B cell stimulation.

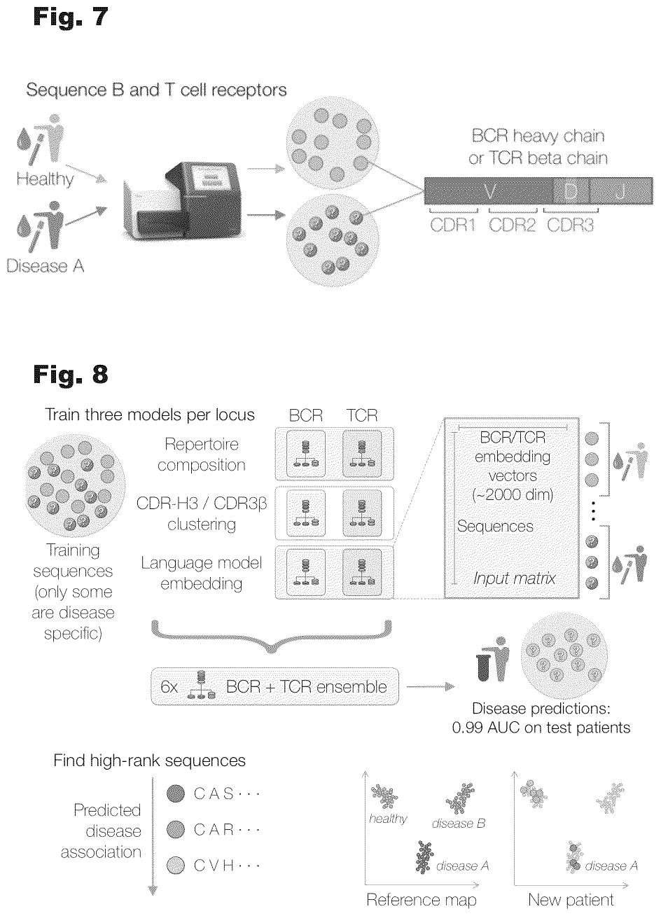

Here, a combination of three models were used per gene locus to improve recognition of distinct kinds of disease states, and to identify similar receptor sequences selected for binding to disease-related antigens. Each classifier model extracts different aspects of immune repertoires (FIG. 8). The first model uses IGHV or TRBV gene segment frequencies and mutation rates across a person's IgH repertoire. The second predictor identifies groups of highly similar sequences across individuals. The third classifier evaluates a broader proxy for functional similarity, rather than direct sequence identity, to find more loosely related immune receptors with common antigen targets. Disease predictors were trained with each representation. The three BCR and three TCR models are then blended into a final prediction of immune status. The final trained program accepts an individual's collection of sequences from peripheral blood B and T cells as input, and returns a prediction of the probability the person has each disease on record (FIG. 8).

This approach was applied to cohorts of patients with diagnoses of Covid-19, HIV, and Systemic Lupus Erythematosus, and healthy controls. New datasets were combined with ones previously reported, all collected with a standardized sequencing protocol, with minimal batch effects. To evaluate whether the proposed strategy can generalize to new immune repertoires, patients were strictly separated into three training, validation, and testing sets, with each person falling into one test set. Some patients had multiple specimens; all were grouped together for the cross-validation divisions. Separate models were trained for each cross-validation group and report averaged classification performance. As described below, the possibility that demographic differences between cohorts could explain diagnosis accuracy was tested and excluded. Details of the three models follow:

Overall repertoire composition: The first machine learning model uses an individual's IgH and TRB repertoire composition to predict disease status. Other groups have piloted immune status classification using deviations in V(D)J recombination gene segment usage from healthy baseline. Certain V gene segments may be more prevalent among antigen-responding V(D)J rearrangements than the general population of immune receptors. As antigen-specific cells become clonally expanded, the distribution of V gene usage across the repertoire can change. Also, class-switched IgH sequences with low somatic mutation (SHM) frequencies were previously identified in acute Ebola or Covid-19 cases, consistent with naive B cells recently having class-switched during the response to infection. These features may also represent repertoire changes accumulated in chronic conditions. A lasso linear model was trained with V/J gene counts and somatic hypermutation rate as features.

Convergent clustering of antigen-specific sequences by edit distance: The second classifier detects highly similar CDR3 amino acid sequences shared between individuals with the same diagnosis. The CDR3s are the highly variable regions of IgH and TRB that often determine antigen binding specificity. For each locus, CDR3 sequences were clustered with the same V gene, J gene, and CDR3 length, and high sequence identity—but allowing for some variability created by somatic hypermutation in B cell receptors. A new sample's sequences can then be assigned to nearby clusters with the same constraints. Clusters enriched for sequences from subjects with a particular disease were selected. These clusters represent convergent sequences that may be predictive of a specific disease across individuals. Each sample's sequences was assigned to these predictive clusters. For each sample, clusters associated with each disease were matched were counted, and these counts were used as features in a lasso linear model to predict immune status.

Language model feature extraction from B and T cell receptor sequences: Amino acid edit distance may not be an optimal measure of receptor similarity. Immune receptor sequences encode complex three-dimensional structures, and small sequence changes can cause important structural changes, while different structures with divergent primary amino acid sequences can bind the same target antigen. While disease-associated receptors may have lexically dissimilar sequences, they may still share the function of binding to the same target. Using language models fine-tuned on BCR and TCR sequences, the third classifier aims to map primary amino acid sequences into a lower-dimensional space that better captures functional similarities, not just the lexical proximity represented by edit distance. Extending beyond prior research using amino acid biochemical properties, like charge and polarity, rather than edit distance alone to find receptor groups, a putative functional representation of BCRs and TCRs were extracted. To do so, UniRep, a self-supervised protein language model, was used to learn functional properties for prediction tasks with an approach adapted from natural language processing. Much like words are the building blocks arranged by grammatical rules to convey meaning, protein sequences are built from amino acids composed in an order compatible with polypeptide chain folding and assuming a structure that can carry out functions, like binding to another molecule or catalyzing a chemical reaction. UniRep was trained to predict randomly masked amino acids using the unmasked amino acids in the remaining sequence context of each protein. This requires learning the short and long-range relationships between different regions of a sequence, analogous to learning natural language phrases and grammar rules to anticipate the next word in a sentence. To achieve its task, the UniRep recurrent neural network compresses each sequence into an internal, low-dimensional embedding, capturing traits that allow accurate reconstruction. If the final model can successfully un-mask protein sequences, the compression and uncompression has extracted fundamental features that summarize the input sequences. UniRep's internal representation was shown to encode fundamental properties like structural classes.

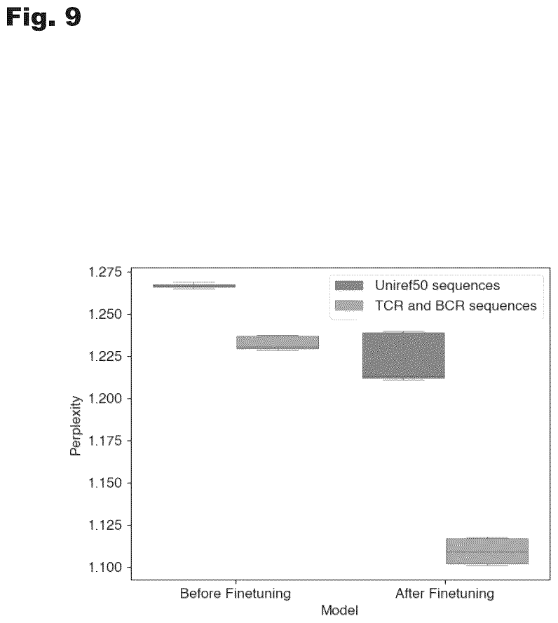

UniRep was originally trained on over 20 million proteins from many organisms. It was hypothesized that by creating a version specialized for immune receptor proteins, improved representations for immune repertoire classification would be obtained. UniRep's training procedure was continued to better reconstruct masked B or T cell receptor sequences. While prior autoencoder models have enabled classification of clusters of similar sequences, the fine-tuned language model approach combines knowledge of global patterns in proteins from many domains of life, with the specific intricacies of BCR and TCR variation; indeed, it was confirmed that the fine-tuned language models retain high performance on UniRep's original training data (FIG. 9). For the disease classification task, the low-dimensional embedding learned by the BCR or TCR fine-tuned language model was used to transform each sequence into a 1900-dimensional numerical feature vector, regardless of sequence length. Then a lasso linear model that maps receptor sequence vectors to disease labels was trained. By aggregating each sequence's predicted class probabilities using a trimmed mean calculation, the model yielded patient-level predictions of specific disease exposures. The trimmed mean was chosen because it is a central estimate robust to noisy contamination by rare sequences with extremely high or low probabilities; testing confirmed this decision in the interest of model stability does not harm performance. Because this classifier starts with a predictor for individual receptors, then aggregates sequence calls into a patient-level prediction, it allows interpretation of which sequences matter most for prediction of each disease. Below, it was confirmed that sequences prioritized by the predictor are enriched for disease-specific B and T cells, demonstrating that the language model learns the syntax of immune receptor sequences, despite their enormous diversity.

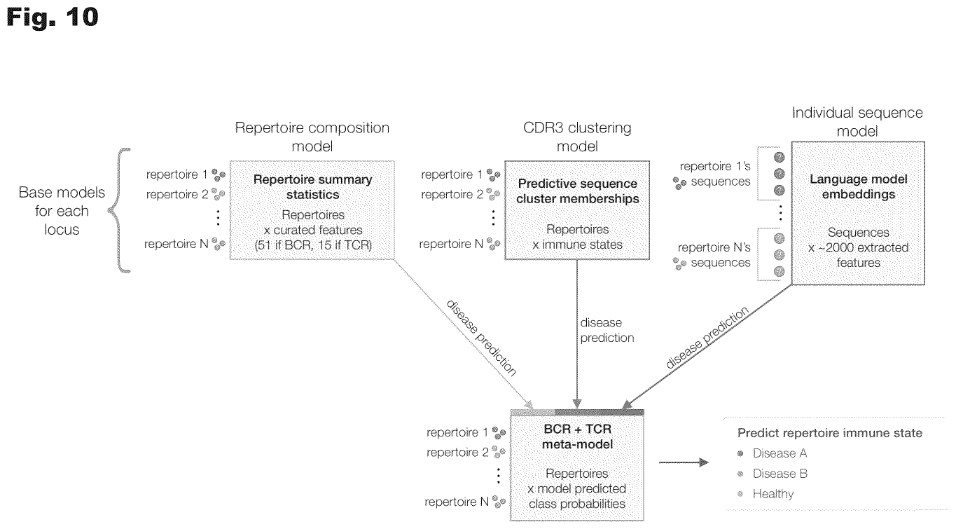

Ensemble: Finally, all three classifiers were combined—the global repertoire composition, CDR3 sequence clustering, and language model embedding strategies—into an ensemble predictor of disease (FIG. 10). This adaptive immune receptor analysis framework was labeled MAchine Learning for Immunological Diagnosis (Mal-ID). By blending the probabilistic outputs from multiple classifiers trained with different strategies, the meta-model exploits each predictor's strengths and can resolve mistakes. (As with the other models, a separate meta-model was trained for each cross-validation group.)

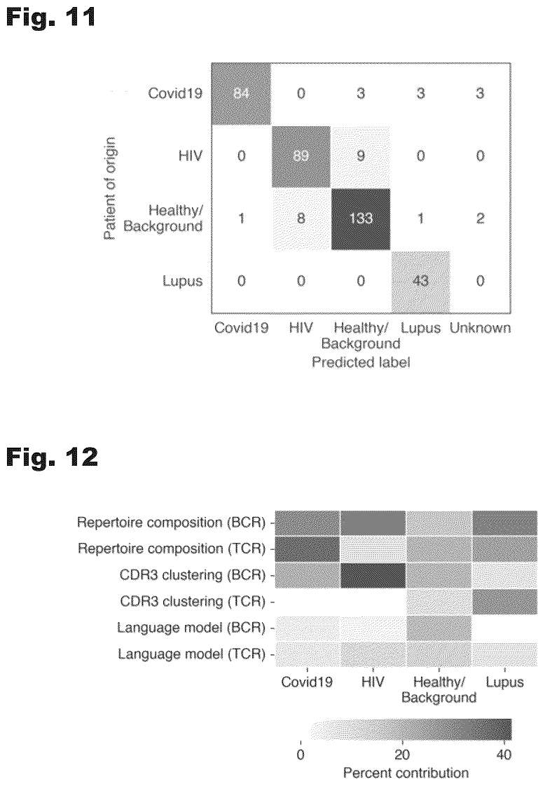

This ensemble approach distinguished five specific disease states in samples from individuals with an area under the Receiver Operating Characteristic curve (AUC) score of 0.99 (FIG. 11). AUC is the likelihood the model ranks a randomly-chosen positive example over a negative example-representing whether the classifier tends to assign high probability to the correct class and low probability to incorrect classes.

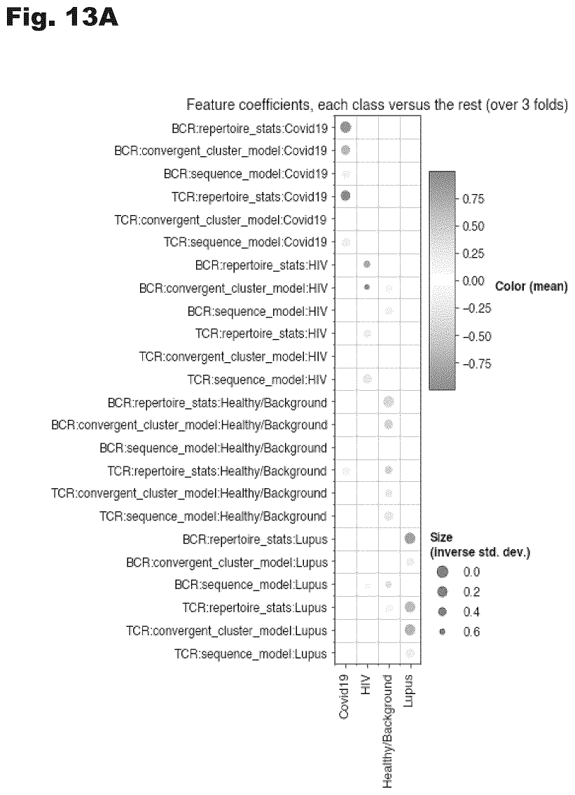

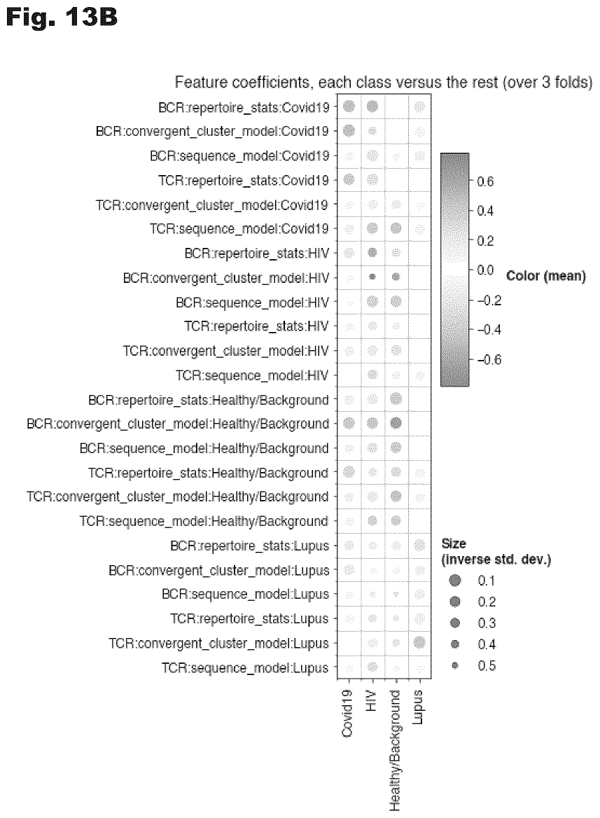

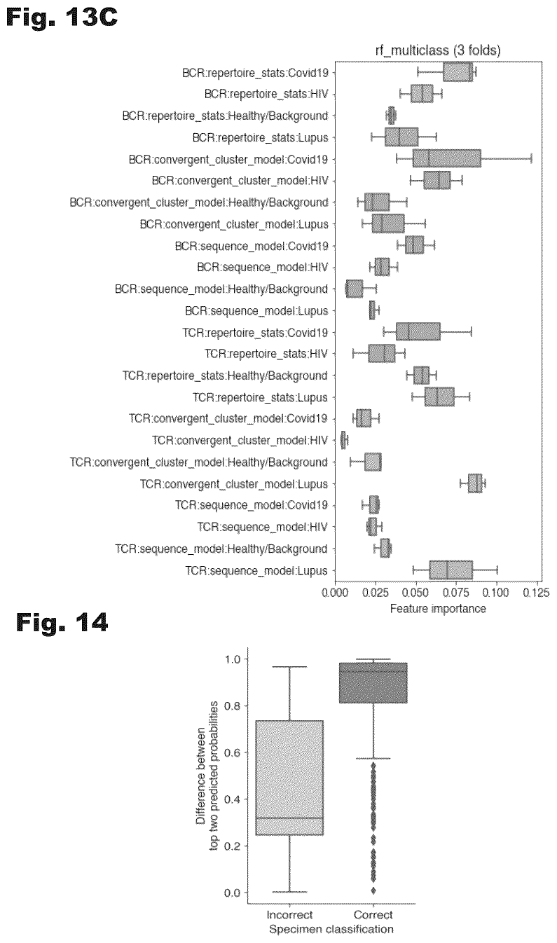

In comparison, the previously-reported CDR3 clustering model, with parallels to many convergent sequence discovery approaches in the literature, achieves only 0.92 AUC for BCR and 0.80 AUC for TCR. To achieve the significantly higher 0.99 AUC in the ensemble approach, all modeling strategies contributed to varying degrees depending on gene locus and disease, suggesting variation in how immune signals are distributed across BCR and TCR repertoires for each disease (FIGS. 12, 13A to 13C). The combined BCR+TCR metamodel performs better than BCR-only or TCR-only versions. The ensemble model achieved 92% accuracy across all held-out test sets.

Of the 8% of misclassified repertoires, 1.3% were samples that did not have any sequences fall into the clonal parameter and edit distance criteria that defined the CDR3 clusters. The CDR3-clustering component of the metamodel abstained from making any prediction for these challenging samples. In the remaining ˜7% of classification mistakes, the ensemble model tended to have low confidence in its prediction (FIG. 14). Allowing the strategy to abstain from inconclusive predictions is important to make the diagnostic robust for challenging real-world cases. In practice, diagnostic sensitivity, the precise threshold on the predicted probability of each disease state, can be tuned to disease prevalence and the desired tradeoff between precision and recall.

While the cross-validation evaluation strategy mitigates the risk of overfitting, it was desired to confirm that the model generalizes to new data from other sources. Covid-19 patient and healthy donor repertoires from other BCR or TCR studies with similar sequencing protocols were assessed. The ensemble model predicted disease type with 100% accuracy in the BCR cohorts and about 95% accuracy in the TCR cohorts. This ability to generalize reinforces that the model has learned true biological signal.

### Limited Impact of Age, Sex, and Race on Classification

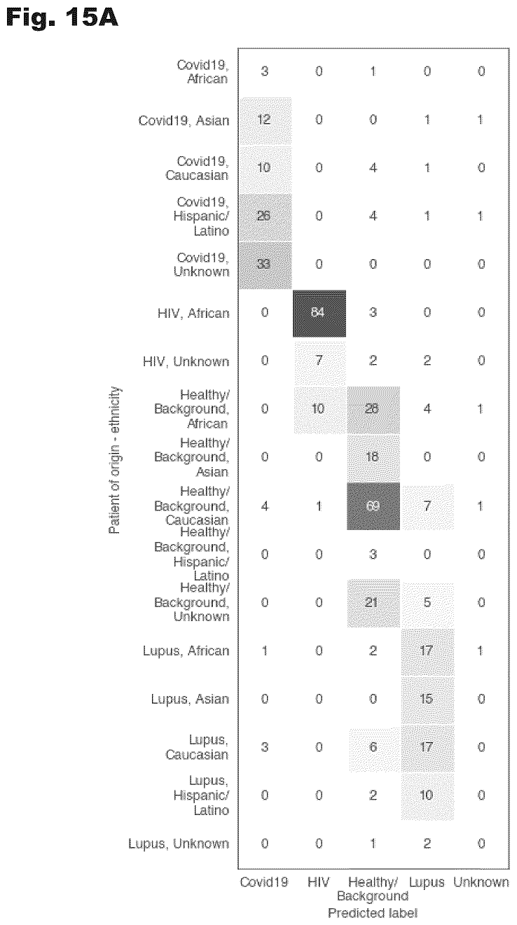

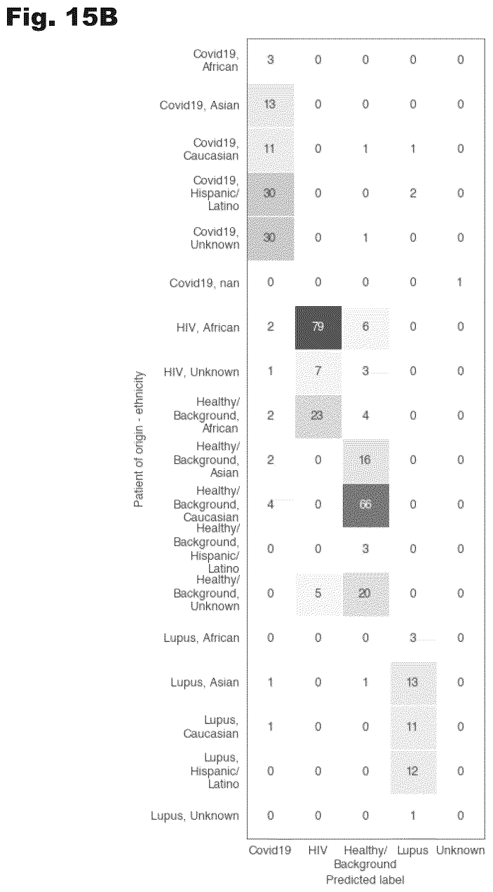

Besides disease, patient demographics also shape the immune repertoire. For example, previous studies have tracked immune aging in gene expression, cytokine levels, and immune cell type frequencies. To study whether extraneous covariates were confounding the disease classification results, it was investigated whether the model could distinguish age, sex, or ancestry of healthy immune receptor repertoires. By training new classifiers to predict these variables, it was found that the sex of a healthy individual could not accurately be determined from IgH or TRB sequences. However, sequences did carry a weak signal of ancestry, with 0.73 AUC predictive power. This signal may have been increased because many individuals with African ancestry in the cohorts live in Africa and have potentially different environmental exposures. A similar pattern was observed in the full disease classification setting, where T cell models poorly distinguished HIV patients and healthy controls from this African cohort, though the corresponding IgH repertoires were distinct (FIGS. 15A and 15B). This corresponds to TCR binding restriction by HLA alleles, which have distinct inheritance patterns in different populations. Accordingly, the metamodel relies more on BCR than TCR signals for HIV prediction (FIG. 12).

Healthy IgH and TRB sequence repertoires also carried a modest signal of age. When age was dichotomized as under or over 50 years old to cast this continuous variable as a classification problem, the prediction model achieved 0.70 AUC. However, the signatures of age detected by the classifier may correspond to different background or environmental exposures for people over 50 versus younger individuals. For instance, circulating influenza virus types have changed following successive pandemics. The first influenza strain someone is exposed to generates a bias in their influenza responses thereafter, likely by forming memory B and T cell pools with specificities related to early virus exposures. When age was divided into groups by decade, the model achieved only 0.62 AUC and abstained from prediction on 12.5% of samples. This worse performance suggests that more granular aging differences are challenging to disentangle at the sequence level with the number of participants, the age ranges, and the cell sampling and sequencing depth in this study. Also, the study was restricted to somatically hypermutated IgD/IgM and class switched IgG/IgA isotypes, reflecting the populations of B cells that are shaped by antigenic stimulation and selection. Studying naive B cells may reveal additional age, sex, or ancestry effects.

It was also investigated whether subtle demographic differences between disease cohorts drove the classification results. For example, the age medians and ranges of the cohorts were: HIV (median 31 years, range 19-64); SLE (median 15 years, range 7-71); healthy controls (median 44 years, range 17-81); Covid-19 (median 48 years, range 21-88). TCR sequences were only available for pediatric patients in the SLE cohort, but this was mitigated by training all BCR models on both pediatric and adult SLE samples (FIGS. 15A and 15B). The percentage of females in each cohort was 51% (healthy controls), 52% (Covid-19), 64% (HIV), and 81% (SLE). The prevalence of females in the SLE cohort matches general epidemiology. The ancestries and geographical locations of participants also differed between cohorts. Most notably, at least 89% of individuals with HIV were from Africa. 63% of individuals known to have Hispanic/Latino ancestry were in the Covid-19 cohort, and 69% of Caucasians were healthy controls.

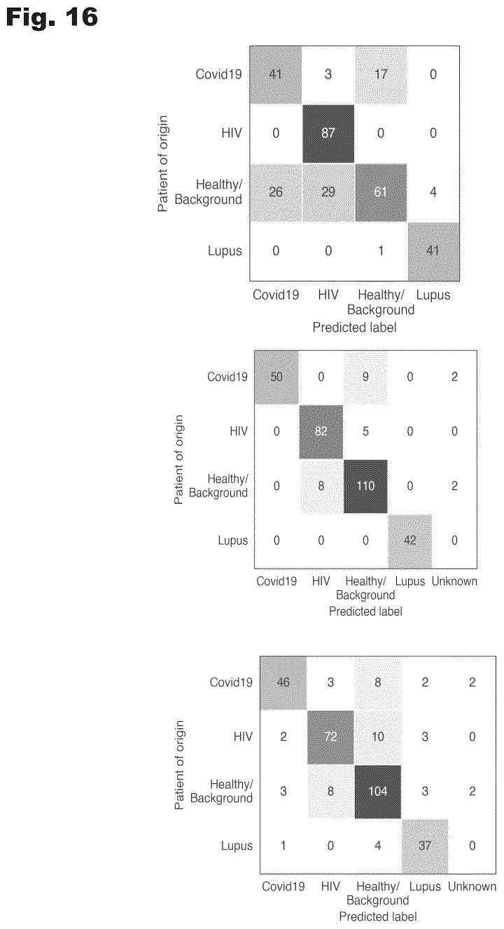

To show that demographic metadata are insufficient to predict disease in the dataset, it was attempted to predict disease state from age, sex, and ancestry alone, without using sequence patterns at all. The demographics-only classifier achieved an AUC of 0.91, substantially lower than the AUC of 0.99 when the sequence prediction ensemble model was retrained with demographic covariates included as features, underscoring how much disease signal was extracted from BCR and TCR sequences (FIG. 16). As an additional version of this test, the disease classification meta-model was also retrained with age, sex, and ancestry effects regressed out from the ensemble feature matrix. After this correction, classification performance on the individuals with full demographic information available dropped slightly from 0.99 AUC to 0.96 AUC (FIG. 16). The small decrement in performance after decorrelating sequence features from demographic covariates suggests that age, sex, and ancestry effects have, at most, a modest impact on disease classification.

### Language Model Recapitulates Immunological Knowledge

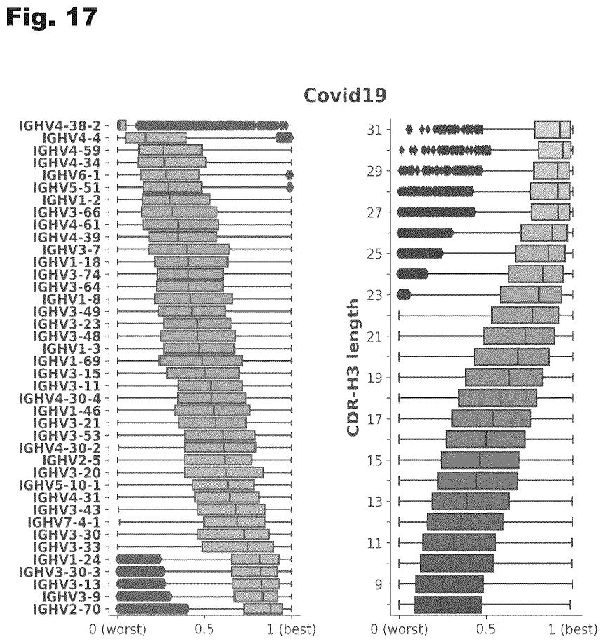

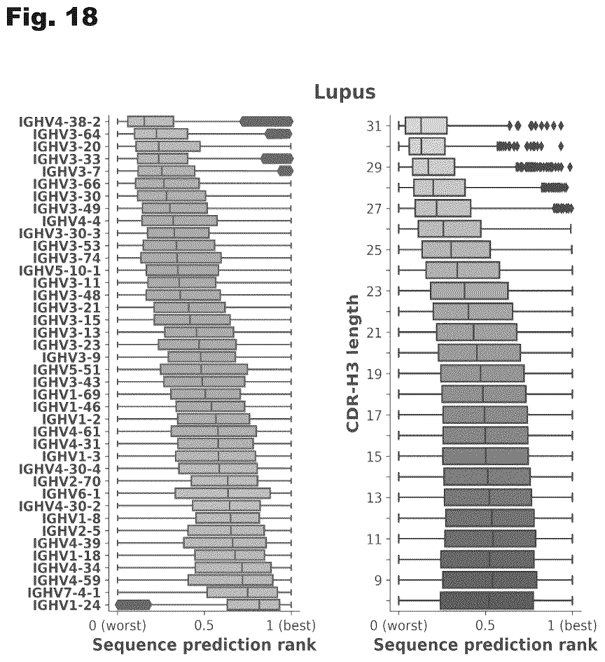

The machine learning framework was designed to identify biologically interpretable features of the immunological conditions, not just provide a black box classifier. To assess the ties between the accurate machine learning classification and known biology, sequences that contributed most to predictions of each disease were examined. For example, all sequences from Covid-19 patients were ranked by the predicted probability of their relationship to SARS-COV-2 immune response using the classifier based on language model embeddings. In discriminating between different diseases, sequences highly prioritized for Covid-19 prediction included IGHV gene segments seen in independently isolated antibodies with strong SARS-COV-2 binding. IGHV3-9 and IGHV2-70 have been implicated in spike protein receptor-binding domain binding, and were highly ranked (FIG. 17). So was IGHV1-24, found in N-terminal domain-directed antibodies. Similarly, the model's prioritization of IGHV4-34, IGHV4-39, and IGHV4-59 for SLE prediction (FIG. 18) matches prior reports that these gene segments are expressed at higher frequencies in SLE patients.

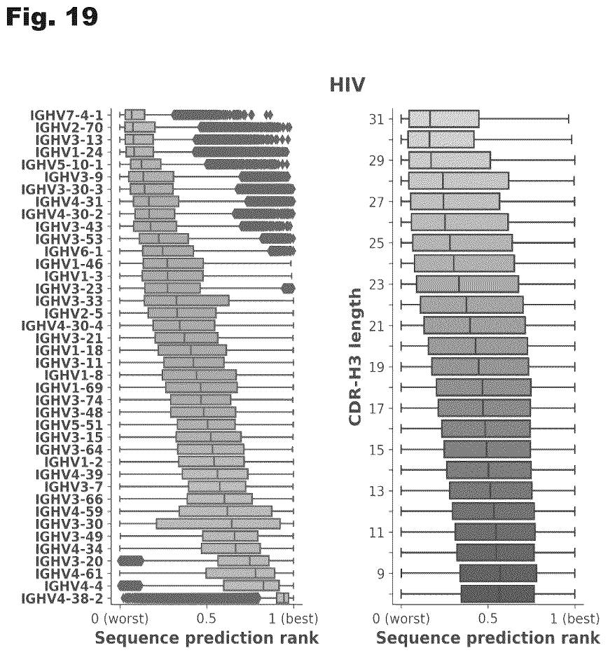

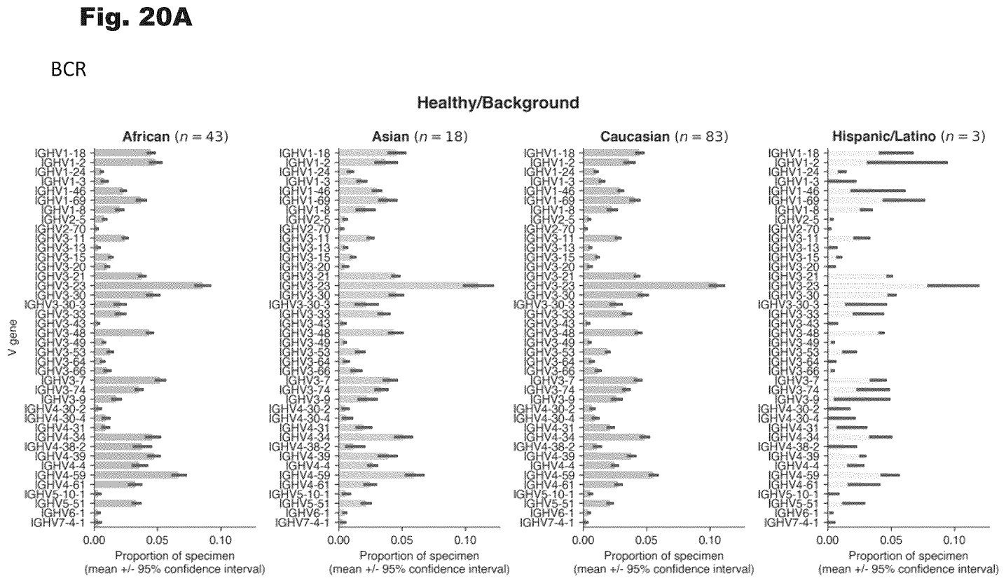

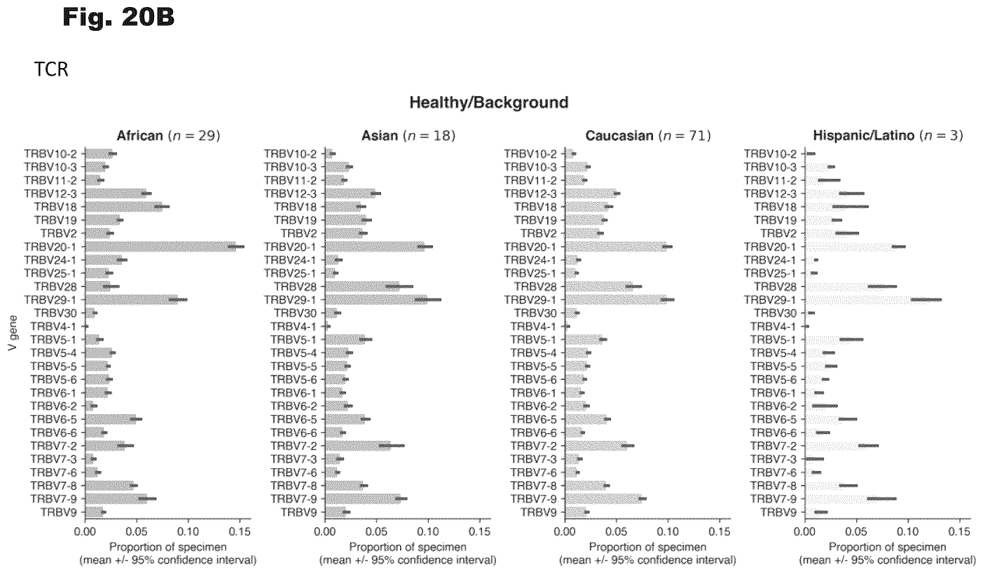

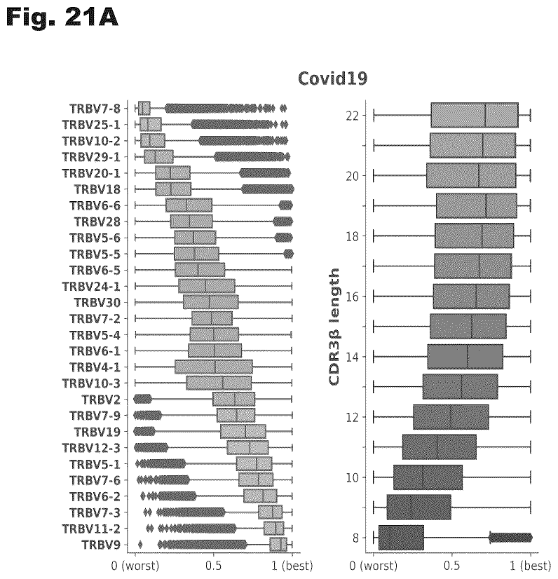

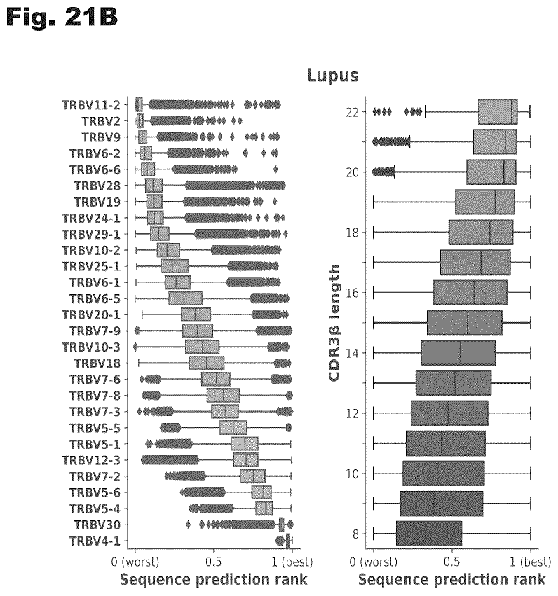

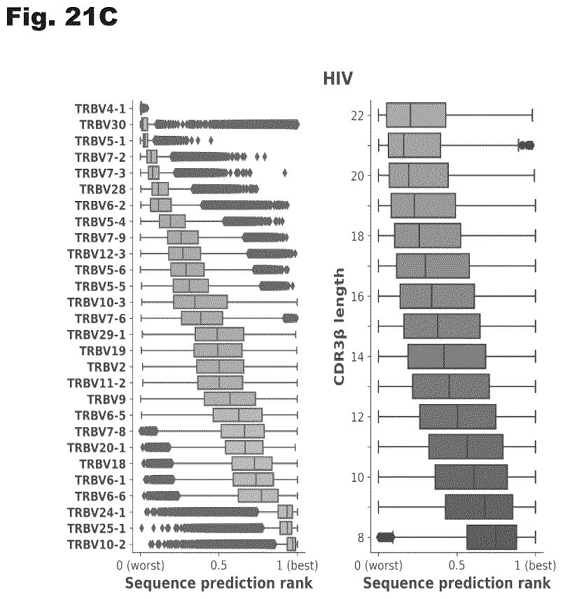

A similar pattern was observed for HIV rankings. IGHV4-34, an IGHV gene previously described in HIV-specific B cell responses—with unusually high somatic hypermutation frequencies in individuals producing broadly-neutralizing antibodies—was ranked highly by the model (FIG. 19). IGHV4-38-2 was also highly ranked for HIV prediction, and was prevalent among HIV-specific B cells. However, IGHV4-38-2 gene usage is significantly more common in African populations in the generated data (FIG. 20A), similar to prior literature. The model may have especially prioritized the IGHV4-38-2 gene because our HIV cohort is predominantly of African ancestry. Other IGHV genes flagged by the model are not stratified by ancestry (FIG. 20A). As expected from HLA allele inheritance patterns that restrict TCR binding, some TRBV genes were also stratified by ancestry (FIG. 20B). TRBV10-2, TRBV24-1, and TRBV25-1, all gene segments enriched in African healthy controls, were the top three highly ranked TRBV gene groups for classifying our predominantly African HIV cohort (FIG. 21B).

The sequence model's rankings also favored certain CDR3 lengths, one of the major features in immunoglobulin and TCR gene rearrangements affected by selection. This was notable, because there is no direct input into the model of raw CDR3 sequences or their length; all UniRep embedding vectors provided as input to the model have identical sizes, regardless of original sequence lengths. Shorter IgH CDR3 lengths were favored by the model for the chronic diseases SLE and HIV (FIGS. 18 and 19), consistent with selection for B cell receptors with shorter CDR3 segments in HIV. On the other hand, IgH sequences with longer CDR3 lengths were favored by the sequence model for Covid-19 class prediction (FIG. 17). These prioritized sequences could reflect B cell clones recently derived from naive B cells that have not yet undergone selection favoring shorter CDR3 lengths in memory B cells. TCR rankings follow the same pattern, except in SLE, where longer CDR3 sequences are favored (FIGS. 21A to 21C).

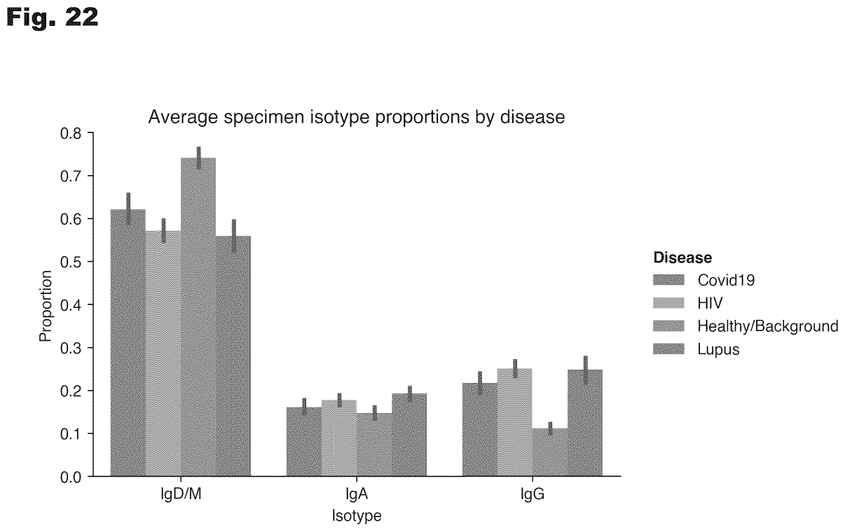

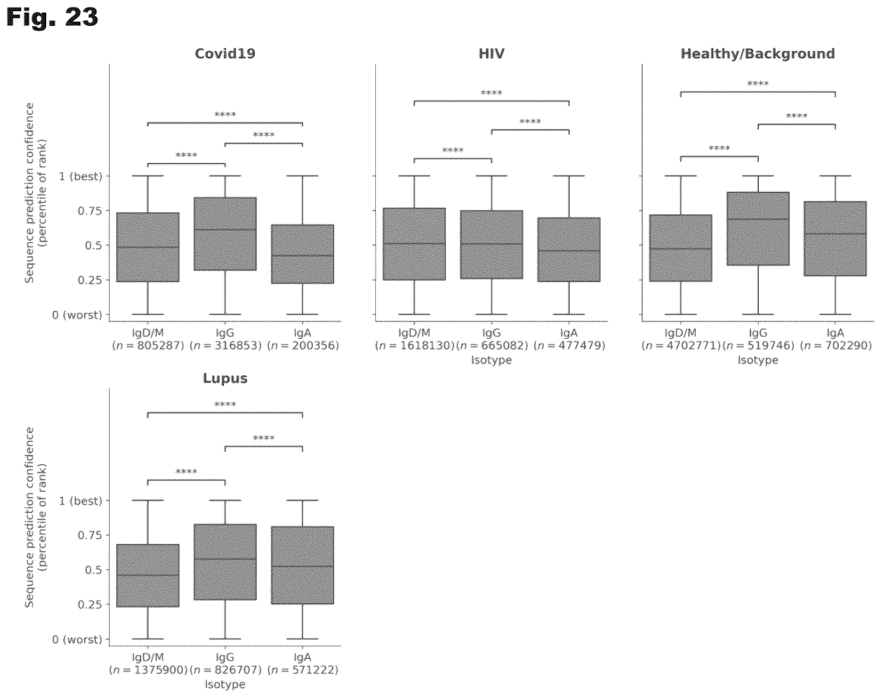

B cell isotype usage varied by person and across disease cohorts (FIG. 22). To prevent isotype sampling artifacts from driving disease predictions, the sequence model was designed to apply balanced weights to all isotypes. As a result, all isotypes were included among model-prioritized sequences for prediction of each disease (FIG. 23). For Covid-19 prediction, IgG sequences played a slightly bigger role than other isotypes, as may be expected in this infectious disease. The other models used in the ensemble were also designed not to be influenced by isotype sampling amounts. The repertoire composition model quantifies each isotype group separately, and the convergent clustering approach is blind to isotype information. To be sure that differences in isotype proportions between patient cohorts were not sufficient to predict disease, a separate model was trained to predict disease from a sample's isotype balance alone—with no sequence information provided. The isotype-proportions model achieved only 0.70 AUC, far lower than the primary model ensemble's 0.99 AUC disease classification performance. Therefore, the classification approach is robust to data artifacts like isotype proportions.

### Language Model Identifies SARS-COV-2 Binders

Only a small minority of peripheral blood B and T cell receptor sequences from Covid-19 patients are directly related to the antigen-specific immune response to SARS-CoV-2. Other naive and memory cells continue to circulate even during acute illness. The 0.99 AUC performance suggests that the ensemble model addresses this “needle in the haystack” issue. The sequences selected by the language model classifier were inspected to assess how important sequences are prioritized.

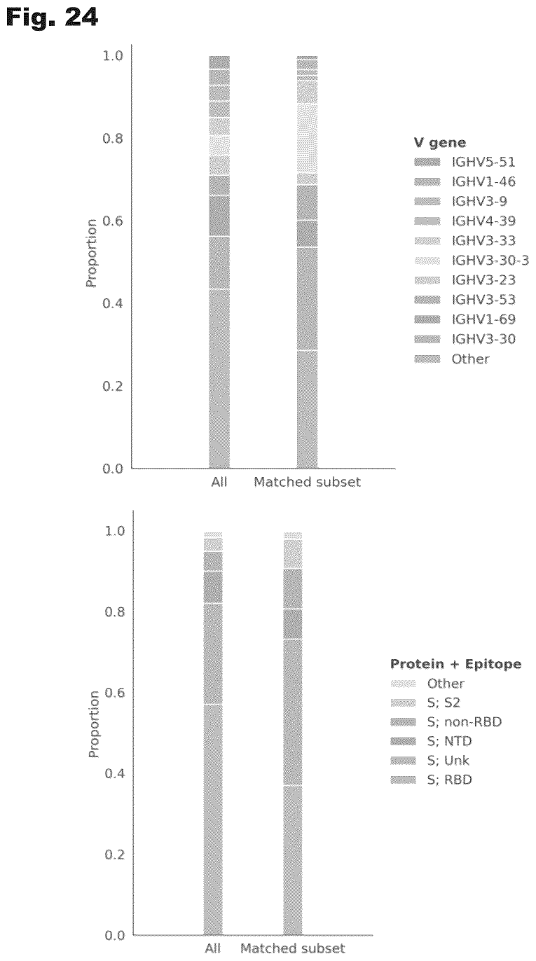

Covid-19 patient sequences can be matched to their nearest neighbors in the databases of SARS-COV-2 specific antibodies and T cells collected by orthogonal experimental methods, such as direct isolation of B cells that bind the SARS-COV-2 receptor binding domain (RBD) followed by BCR sequencing. Unlike the global repertoire scans of a limited number of patients, the external databases include larger source cohorts, meaning they may contain more Covid-19 response types than this dataset. The BCR database is also biased towards potential therapeutic antibodies identified by isolating spike antigen-specific B cells. Despite these differences, sequences from the Covid-19 cohort had high sequence identity matches to over 9% of known binding antibodies in the CoV-AbDab database, covering all major epitopes and IGHV genes (FIG. 24). 63% of the matching BCR sequences in the dataset were IgG sequences, followed by IgD/M (20%) and IgA (7%), with the final 10% seen in multiple isotypes. This IgG dominance pattern reflects how sequences that class switched to IgG were stimulated by antigen, and is consistent with the isotype relationships examined above. As a negative control, the process was repeated with sequences from healthy subjects. Healthy donor-originating sequences matched 5.4% of CoV-AbDab clusters in total, representing an expected decrease in CoV-AbDab matches. Over 93% of the matched healthy control sequences came from the IgD/M isotypes, most likely representing naive B cells that could mount a response should SARS-COV-2 enter the body. Matches were rare: 0.14% of unique Covid-19 patient sequences from the dataset matched any CoV-AbDab cluster, along with 0.01% of unique healthy subject sequences. This order of magnitude difference is expected because antigenic stimulation of the IgG population in Covid-19 patients results in clonal expansion and diversification through somatic hypermutation.

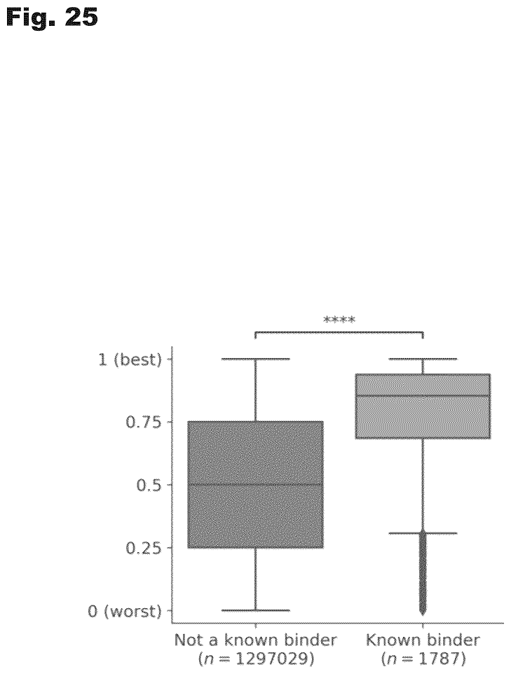

In support of the biological relevance of the model's top-ranked sequences, many were independently validated to complement SARS-COV-2. Covid-19 patient-originating sequences that overlapped with this known-binder database were assigned significantly higher ranks by the prediction model (FIG. 25). When viewed as how well the model discovered known binder BCRs with model rankings, an AUC of 0.775 was achieved, and 87% of matches occurred in the top half of ranked BCR sequences, respectively. These binding relationships were not known to the classifier at training time, and CoV-AbDab sequences were not used to train the model. The concordance between automatically-prioritized sequences and experimentally-validated, disease-specific sequences from separate cohorts suggests the language model classifier learned meaningful rules that recapitulate biological knowledge gained during the extraordinary international research effort in response to the COVID-19 pandemic.

These known-binder discovery results were compared to an alternative strategy representative of common approaches to find convergent disease-specific BCR or TCR patterns. Known binders were sought among any Covid-19 patient sequence that fell into a Covid-19 BCR cluster identified by the CDR3 clustering model. In total, these sequences matched only 0.65% of BCR known binders—a fraction of the total set of known binders that could be discovered in the patient cohorts. This result demonstrates that the language model approach to disease classification can be applied to discover far more antigen-specific sequences than the mainstay method in the field.

### Repertoire Progression Between Disease States

To evaluate further disease-specific insights from the model, a novel immune repertoire visualization was developed to convey disease status at a glance. From the training set, a reference two-dimensional UMAP layout was created using receptors that the language model classifier learned to confidently separate into distinct groups by immune state. Since this supervised UMAP is conditioned on the disease labels assigned to sequences, any visual distortions created by the reduction into two dimensions are less likely to bias against the disease classes.

Sequences that were held out from the training set were overlaid onto the reference UMAP visualization. For example, monoclonal antibodies can be evaluated using the language model's interpretation. Therapeutic monoclonal antibodies against SARS-COV-2 can be visualized based on where they fall into the language model's representation.

With the same visualization technique, repeated samples can be projected from a held-out test set patient onto the reference map, enabling immune repertoire composition monitoring over time. Patient repertoires contain a multitude of high-confidence and low-confidence sequences for disease prediction. Sequences with low probability predictions by the model can be excluded to focus the visualization on BCRs likely to be disease-specific. As an example Covid-19 patient's infection and immune responses progress, the visualization may reveal their collection of immune receptors shifting from a Healthy/Background region soon after onset of symptoms, into a Covid-19 area later.

### Methods Supporting the Data

### B and T Cell Repertoire Sequencing

Immune receptor were assembled repertoires from 69 Covid-19, 95 chronic HIV-1, and 66 Systemic Lupus Erythematosus (SLE) patients, along with 168 healthy controls. Mild Covid-19 cases and samples prior to seroconversion were excluded. These filters limited model training data to peak-disease samples to improve the chances of learning patterns for the disease-specific minority of receptor sequences. However, it was desired to avoid creating an artificially simple classification problem from filtering to trivially separable immune states. To this end, the HIV cohort included patients regardless of whether they generated broadly neutralizing antibodies to HIV. If the analysis was restricted to HIV-infected individuals who produce broadly neutralizing antibodies, a more-easily separable HIV class may have been created, due to the unusual characteristics of those antibodies.

Across these diverse immune states, millions of B and T cell receptors were sampled, PCR amplified with immunoglobulin and T cell receptor gene primers, and sequenced. Briefly, T cell receptor beta chains and each immunoglobulin heavy chain isotype were amplified in separate PCR reactions using random hexamer-primed cDNA templates, and paired-end Illumina MiSeq sequencing was performed. To reduce the potential for batch effects, data collection followed a consistent protocol. V, D, and J gene segments were annotated with IgBLAST v1.3.0, keeping productive rearrangements only. Using IgBLAST's identification of mutated nucleotides, the fraction of the IGHV gene segment that was mutated in any particular sequence was calculated; this is the somatic hypermutation rate (SHM) of that B cell receptor heavy chain. The dataset was restricted to CDR-H3 and CDR3B segments with eight or more amino acids; otherwise the CDR3 clustering method below might group short but unrelated sequences.

Then nearly identical sequences were grouped within the same person into clones. For each individual, all nucleotide sequences from all samples (including samples at different timepoints) across all isotypes were grouped, and ran single-linkage clustering, requiring clustered sequences to have matching IGHV/TRBV genes, IGHJ/TRBJ genes, and CDR-H3/CDR3β lengths, and at least 90% CDR-H3 suitable CDR3β sequence identity by string substitution distance. Among the BCR sequences, only class-switched IgG or IgA isotype sequences, and non-class-switched but still antigen-experienced IgD or IgM sequences with at least 1% SHM were kept. By restricting the IgD and IgM isotypes to somatically hypermutated BCRs only, any unmutated cells that had not been stimulated by an antigen and were irrelevant for disease classification were ignored. The selected non-naive IgD and IgM receptor sequences were combined into an IgM/D group. Finally, the dataset was deduplicated. For each sample from a patient, one copy of each clone per isotype was kept—choosing the sequence with the highest number of RNA reads. Similarly, one copy of each TCRβ clone was kept. On average, any two patients had 0.0005% IgH and 0.167% TRB sequence overlap, underscoring the enormous diversity of T cell receptor and especially B cell receptor sequences.

### Cross Validation

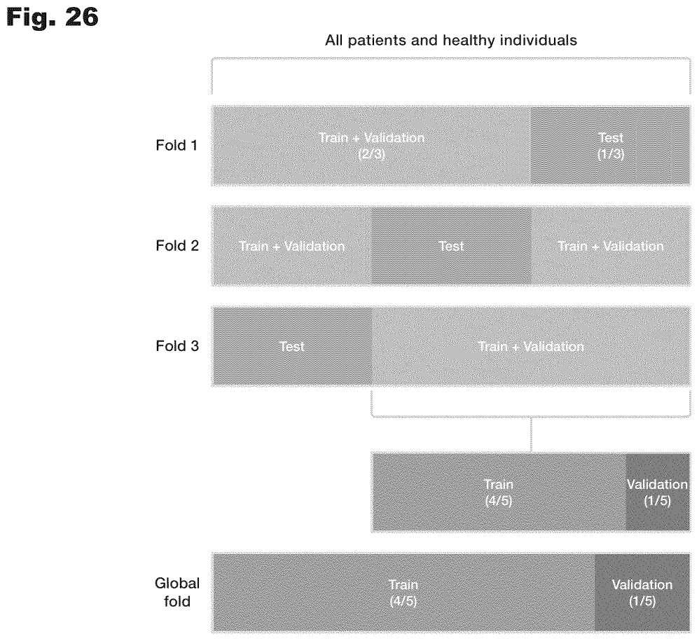

Individuals were divided into three stratified cross-validation folds, each split into a training set and a test set (FIG. 26). The splits were respected across the training of the complete pipeline. Stratified cross-validation preserved the global imbalanced disease class distribution in each fold. A validation set was carved out from each training set, to use for several tasks described below: language model fine-tuning, classifier hyperparameter optimization, and ensemble metamodel training. In all folds, less than 0.1% of sequences shared between any pair of the train, validation, and test sets was observed. Since any single repertoire contains many clonally related sequences, but is very distinct from other people's immune receptors, all sequences from an individual person was placed into only the training, validation, or the test set, rather than dividing a patient's sequences across the three groups. Otherwise, the prediction strategies evaluated here could appear to perform better than they actually would on brand-new patients. Given the chance to see part of someone's repertoire in the training procedure, a prediction strategy would have an easier time of scoring other sequences from the same person in a held-out set. This prevented overfitting of the models to the particularities of training patients.

### Evaluation Metrics

Models were trained with the scikit-learn implementations of random forests, support vector machines, and logistic regression with lasso regularization and multinomial loss, using balanced class weights and default hyperparameters. Predicted labels from all test sets were concatenated for global accuracy evaluation. On the other hand, performance metrics that take predicted class probabilities as input, including ROC AUC and auPRC, were computed separately for each fold, because probabilities may be on different scales in each fold and should not be combined for a global AUC or auPRC score. We report multiclass AUC and auPRC calculated in a one-vs-one fashion, taking the class size-weighted average of the binary AUCs/auPRCs calculated for each pair of classes, allowing each class a turn to be the positive class in the pair. All analyses were performed and plotted with python v3.9.13, numpy v1.22.0, pandas v1.4.3, scipy v1.8.1, scikit-learn v1.1.1, jax v0.3.14, umap-learn v0.5.3, matplotlib v3.5.2, and seaborn v0.11.2.

### Disease Classifier Using Overall Repertoire Composition Features

For each sample, IgG, IgA, IgM/D, and TRB summary feature vectors were created by tallying IGHV/TRBV gene and IGHJ/TRBJ gene usage, counting each clone once. To account for different total clone counts across samples, total counts were normalized to sum to one per sample. Then log-transformation and Z-scoring (i.e. subtracted the mean and divided by the standard deviation, to achieve zero mean and unit variance) were performed on the matrix representing how counts are distributed across V-J gene pairs. Finally, a PCA was performed to reduce the count matrix to fifteen dimensions. All transformations were computed on each training set and applied to the corresponding test set. In addition, for each sample's subset of BCR sequences belonging to each isotype, the median sequence somatic hypermutation rate and the proportion of sequences that are somatically hypermutated (with at least 1% SHM) was calculated. Only BCRs have somatic hypermutation, so mutation rate features of TCRs were not included. In total, the IgH model arrived at 51 features across IgG, IgA, and IgM/D (fifteen count PCs and two mutation rate features per isotype), and the TRB model arrived at 15 features.

Separate lasso logistic regression linear models with L1 regularization were fit on the 51-dimensional (17×3 isotypes) BCR and 15-dimensional TCR feature vectors from each sample to predict disease. Features were standardized to zero mean and unit variance. This feature engineering and model training procedure was repeated on each cross-validation fold separately, then results were combined from all test folds.

Disease Classifier by Clustering CDR-H3 Sequences with Edit Distance

Single-linkage clustering was performed on CDR3B sequences from T cells with identical TRBV genes, TRBJ genes, and CDR3B lengths, and separately on CDR-H3 sequences from B cells with identical IGHV genes, IGHJ genes, and CDR-H3 lengths. Nearest-neighbor clusters were iteratively merged if all cross-cluster pairs had high sequence identity, as measured by string substitution distance.

Filter to BCR and TCR disease-specific clusters: clusters with sequences from three or more individuals were kept, as long as at least 80% of those individuals were positive for some disease. For each remaining predictive cluster, a cluster centroid was created—a single consensus sequence. Recall that each cluster member is a clone from which only the most abundant sequence was sampled. Rather than having each cluster member contribute equally to the consensus centroid sequence, contributions at each position were weighted by clone size: the number of unique BCR or TCR sequences originally part of each clone.

Compute BCR and TCR feature vector for each sample: Sequences from a sample were then matched to these predictive cluster centroids. In order to be assigned, a sequence must have the same IGHV/TRBV gene, IGHJ/TRBJ gene, and CDR-H3/CDR3β length as the candidate cluster, and must have at least 85% (BCR) or 90% (TCR) sequence identity with the consensus sequence representing the cluster's centroid. After assigning sequences to clusters, cluster memberships was counted across all sequences from each sample. These cluster memberships were found for training set samples, then computed a feature vector for each sample. A sample's score for a particular disease was defined as the number of disease-predictive clusters into which some sequences from the sample were matched. This featurization captures the presence or absence of convergent T cell receptor or immunoglobulin sequences (separated by locus, but without regard for BCR isotypes).

Fit and evaluate model for each locus: Features were standardized, then used to fit separate BCR and TCR linear logistic regression models with L1 regularization and balanced class weights (inversely proportional to input class frequencies). The featurizations and models were fitted on each training set and applied to the corresponding test set.

If a sample had no sequences fall into a predictive cluster, no prediction was made. These abstentions hurt accuracy scores, but were not included in the AUC calculation, since no predicted class probabilities are available for abstained samples. Fewer than 1.5% of samples resulted in abstention.

### Language Model Representations for Immune Sequences

The CDR-H1/CDR1β, CDR-H2/CDR2β, and CDR-H3/CDR3β segments of each receptor sequence were combined, then the concatenated amino acid strings were embedded with the UniRep neural network, using the jax-unirep v2.1.0 implementation. A final 1900-dimensional vector representation was calculated by averaging UniRep's hidden state over the original protein's length dimension.

To embed sequences, weights fine-tuned on a subset of each cross-validation fold's training set were used, yielding a total of six fine-tuned models: one per fold and gene locus. The weights that minimized cross-entropy loss on a subset of the held-out BCR or TCR validation set were chosen. For example, UniRep was fine-tuned on fold 1's BCR training set until reaching minimal cross-entropy loss on fold 1's BCR validation set.

The fine-tuning procedure was unsupervised. Besides the raw CDR1+2+3 sequence, no disease or other class labels were provided during fine-tuning. As a result, the fine-tuned language models are specialized to B or T cell receptor patterns, but not hyper-specialized to the disease classification problem. They can be applied to other immune sequence prediction tasks. During the fine-tuning process, cross-entropy loss on the B or T cell validation set drops as expected, and importantly, the cross-entropy loss does not increase on UniRep's original Uniref50 dataset. This result confirms that fine-tuning does not cause catastrophic forgetting of UniRep's own training data, meaning the final language models retain knowledge of general protein patterns in addition to B or T cell receptor specific information.

### Disease Classifier Using Language Model Embeddings

The analysis pipeline for classifying disease with language model embeddings of sequences is complex, but necessarily so because it aggregates individual sequence data to generate patient-level predictions.

Sequence-level disease classifier: First, lasso classification models were trained to map sequences to disease labels-one model per fold and per locus. As input data, fine-tuned UniRep embeddings (standardized to zero mean and unit variance) were used, along with categorical dummy variables representing the IGHV gene and isotype of each BCR sequence or the TRBV gene of each TCR sequence.

Making predictions for individual sequences before aggregating to a patient-level prediction has interpretation benefits, but the two-stage approach introduces a new challenge. The available ground truth data associates patients, not sequences, with disease states. It is not known which of their sequences are truly disease related. To train the individual-sequence-level model, noisy sequence labels derived from patient global immune status were provided. But this transfer creates very noisy labels: even at the peak-disease timepoints in the dataset, disease-specific immune receptor patterns nevertheless represent just a small subset of a patient's vast immune receptor repertoire. Unreliable sequence labels are to be accounted for and the right subset of sequences are chosen to make a patient-level decision.

Highly regularized statistical models equipped to withstand the noisy training labels created by transferring patient labels to the sequence-level prediction task were used. The lasso's L1 penalty encouraged sparsity among the ˜2000 input features. Because isotype use varies from person to person, the sequence-level BCR model was trained with isotype weights to account for this imbalance.

Aggregate sequence predictions to sample prediction: Since there were no true sequence labels, classification performance cannot be evaluated for the sequence-level classifier. Instead, BCR or TCR sequence predictions were aggregated into a patient sample-level prediction. Using the predicted disease class probabilities for each sequence belonging to a sample, the trimmed mean was computed for each class across the sequences. That is, the top and bottom 10% of outlying scores were removed, then the mean of the remainder was computed, weighing sequences inversely proportional to their isotype's overall usage in the sample. (This way, minority isotype signal is not drowned out.) Then disease class probabilities were renormalized to sum to one for each sample.

Tune class decision thresholds: To complete these BCR and TCR sample-level classifiers based on aggregating sequence predictions, class decision thresholds were tuned against the held-out validation set. Specifically, class probabilities were reweighted to optimize the Matthews correlation coefficient, a classification performance metric that is meaningful even under class imbalance. Before applying class weights, the winning label for each sample was chosen based on the class with highest predicted probability. If a class then had its probabilities reweighted by ⅕, for example, the model must be five times more confident to choose that class label. Importantly, these weights were applied only in the choice of a final predicted label for each sample. This procedure affected the confusion matrix, accuracy, and other metrics based on predicted labels, but the AUC did not change. It was reasoned that this adjustment is necessary for fair evaluation of the language model classifier strategy, since the mean-of-each-class sequence prediction aggregation strategy, followed by a renormalization to sum to 1, does not necessarily produce calibrated probabilities. The tuned-decision-thresholds model versions were only used to evaluate the BCR and TCR language model components on their own. On the other hand, the original class probabilities were not reweighted before entering the ensemble metamodel feature matrix.

Evaluate classifier: Finally, the sequence-prediction-aggregating predictor was evaluated on the test set. Each test sample's sequences were scored, then combined with a trimmed mean as above. The resulting disease class probabilities for each sample were reweighted by the global class weights found above, to arrive at final predicted sample labels. Ground truth sample disease status is known, so classification performance could be evaluated, unlike at the sequence-level prediction stage.

### Ensemble Metamodel

After training repertoire composition, CDR3 clustering, and language model embedding and aggregation models on each fold's training set, the classifiers were combined with an ensemble strategy. For each fold, all trained base classifiers were run on the validation set, and the resulting predicted class probability vectors from each base model were concatenated. Any sample abstentions from the CDR3 clustering model were carried over (the other models do not abstain). Finally, a new lasso logistic regression classification model was trained to map the combined predicted probability vectors to validation set sample disease labels. The model was trained in a “one-vs-rest” fashion. This metamodel was evaluated on the held-out test set.

Having integrated many datasets in this study, it was to be confirmed that the disease classification performance is not driven by technical differences between batches. It would be expected in any study of human cohorts to identify some degree of batch effects, given the difficulty of collecting identical samples in identical manner, at identical severity and timepoints, from patients suffering from diseases that appear in different populations at different frequencies.

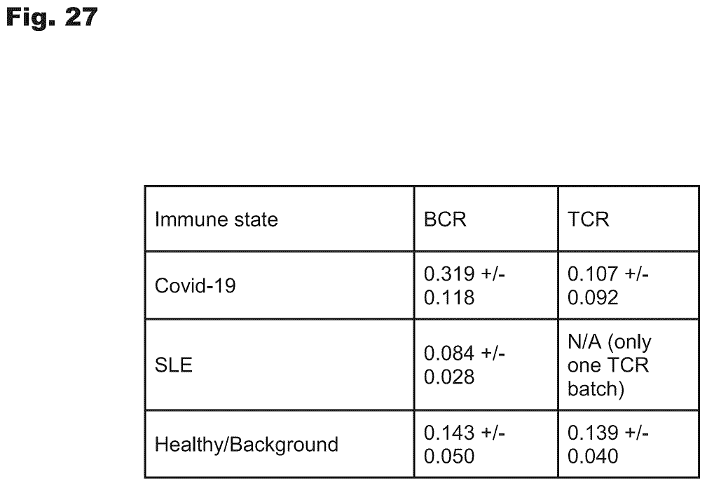

Batch differences can be evaluated using the language model embeddings of BCR and TCR repertoires from the disease types found in multiple batches, for example for Covid-19 patients, SLE patients, and healthy donors. The kBET batch effect metric from the single cell sequencing literature can be applied. kBET measures whether cells from many batches are well-mixed by comparing the batch label distribution among each cell's neighbors to the global distribution. In place of cells described by gene expression vectors, sequences described by language model embedding features were assessed. kBET was measured for every disease in every test set fold and in both BCR and TCR data. For example, a k-nearest neighbors graph (k=50) was constructed with all BCR sequences from Covid-19 patients in test fold 1. Chi-squared tests was performed for the difference between the batch label distribution among each sequence's 50 nearest neighbors and the expected distribution from the total number of sequences belonging to each batch in the entire graph. After multiple hypothesis correction with a significance threshold of p=0.05, the number of sequences that could have rejected the null hypothesis that the local neighborhood batch distribution is the same as the global batch distribution were measured. Aggregating these results by disease across gene loci and folds, it was seen that the null hypothesis is rejected for 15.9% of sequences on average, suggesting that the data is well mixed (FIG. 27). The average rejection rate is higher for Covid-19 BCR sequences at 31.9%, which may be influenced by disease severity differences between cohorts. Time point differences between batches may also have an effect on kBET metrics for acute diseases like Covid-19. At earlier time points, Covid-19 patient repertoires may include more healthy background sequences, leading to a different batch overlap graph in comparison to how batches compare after clonal expansion of Covid-19 responding sequences. Overall, the results in these exemplary data suggest that most sequences have well-mixed batch proportions amongst their nearest neighbors.

### Validation on External Cohorts

To further confirm that the model has learned true biological signal as opposed to batch effects, the model's ability to generalize to unseen data from other cohorts was tested. For this purpose, rather than using a model trained on one of the cross-validation divisions of the dataset, a new global model incorporating all data (without holding out a test set) was trained (FIG. 26). A validation set was still held out for the purpose of training the ensemble metamodel, with an equivalent ratio of training set to validation set size as in the cross validation regime. Data from other IgH and TRB repertoire studies with cDNA sequencing were downloaded, reprocessed through IgBLAST to ensure consistent gene nomenclature, then processed through the entire model architecture.

Predicting Demographic Information from Healthy Subject Repertoires

The above process was repeated to predict age, sex, or ancestry instead of disease. Input data was limited to healthy controls to avoid learning any disease-specific patterns. To cast this as a classification problem, age was discretized either into deciles or as a binary “under 50 years old”/“50 or older” variable. Notably, only one healthy control individual was over 80 years old. Therefore, the data do not assess repertoire changes at more extreme older ages. The healthy individual over 80 years old was excluded from the analysis.

For each of the three tasks, the full BCR and TCR model and metamodel architecture was trained on all cross validation folds. Data was not explicitly introduced from allelic variant typing in germline V, D, or J gene segments or in HLA genes into the models, but such data could be expected to increase detection of ancestry in such datasets.

### Evaluating Predictive Power of Potential Demographic Confounding Variables

The entire disease-prediction set of models was retrained on the subset of individuals with known age, sex, and ancestry. (As above, any individuals over 80 years old were excluded.) Additionally, those demographic variables from the feature matrix used as input to the ensemble step were regressed out. Specifically, a linear regression was fit for each column of the feature matrix, to predict the column's values from age, sex, and ancestry. The feature matrix column was then replaced by the fitted model's residuals. This procedure orthogonalizes or decorrelates the metamodel's feature matrix from age, sex, and ancestry effects. Covariates at the metamodel were regressed out stage because it is a sample-level, not sequence-level model, and age/sex/ancestry demographic information is tied to samples rather than sequences.

Separately, models were also trained to predict disease from either age, sex, or ancestry information encoded as categorical dummy variables. Here, no sequence information was provided as input. The best-performing model in each case ranged from a linear SVM, to a linear logistic regression model with elastic net regularization, to a random forest model. Separately, models were also trained to predict disease from sequence features, along with age, sex, and ancestry information, and along with interaction terms that multiply each BCR or TCR sequence feature with each demographic feature. Comparing performance of these models to the demographics-only models shows the added value of adding sequence information.

### Model Ranking of Disease-Specific Sequences

In each test set, Covid-19 patient-originating sequences were scored with the sequence-level classifier based on language model embeddings. Predicted Covid-19 class probabilities were combined for all sequences across folds. Some sequences were seen in multiple people, appearing in more than one test fold and thus receiving a different predicted probability from each fold's model. These sequences were deduplicated by choosing the copy with highest predicted disease class probability, to capture just how disease-related the sequence could be. Then sequences were ranked by their predicted probability, and ranks were rescaled from 0 to 1 (highest original probability). This process was repeated for other diseases.

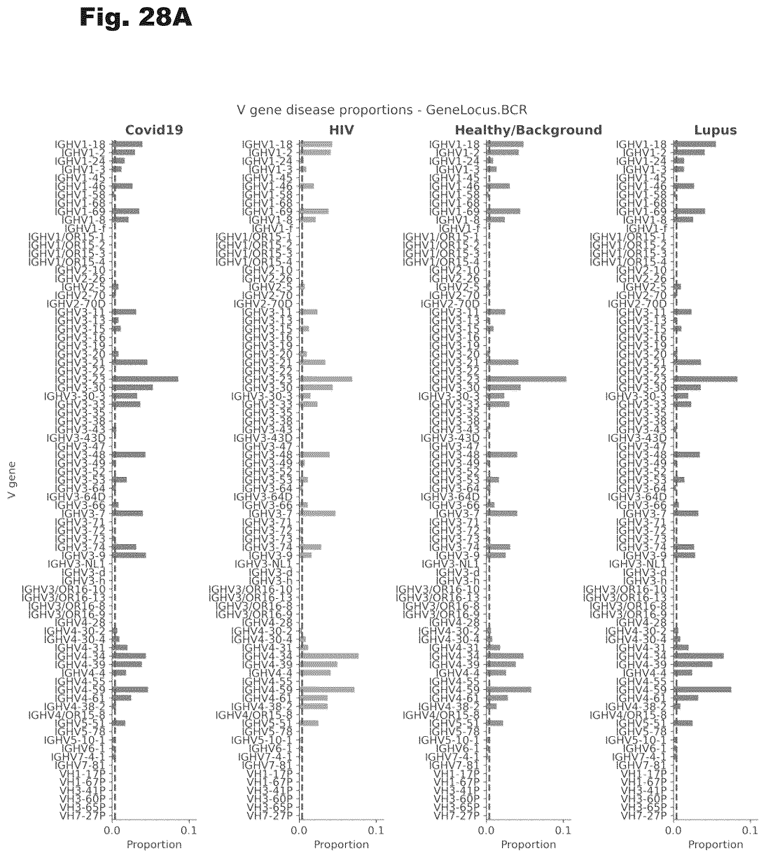

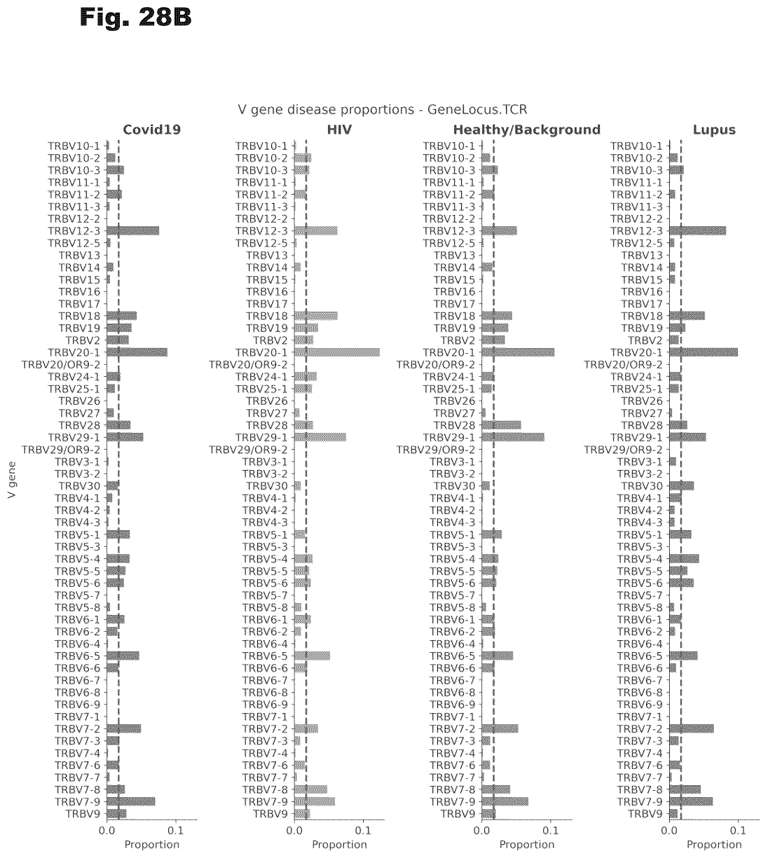

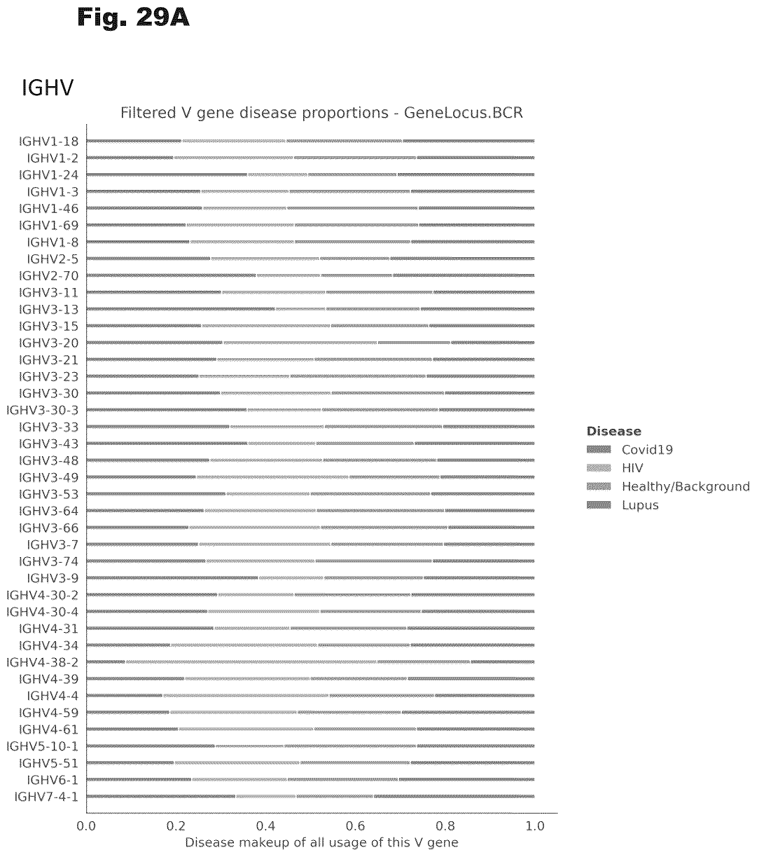

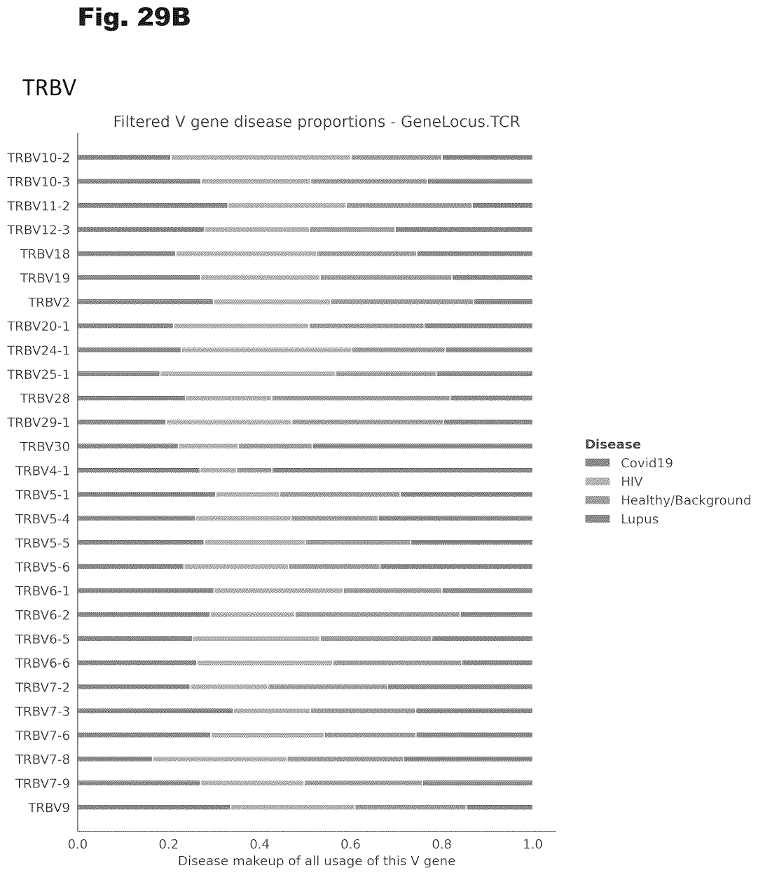

Using these ranked sequence lists, the relationship between rank and sequence properties, like CDR-H3/CDR3β length, isotype, and IGHV/TRBV gene segment, was examined. For the V gene usage comparison, V genes with very low prevalence were removed. To set a prevalence threshold, the greatest proportion each V gene ever comprises of any cohort was found, and the median of these proportions was utilized (FIGS. 28A and 28B). The following rare IGHV and TRBV genes were filtered out (half of the totals): IGHV1-45, IGHV1-58, IGHV1-68, IGHV1-f, IGHV1/OR15-1, IGHV1/OR15-2, IGHV1/OR15-3, IGHV1/OR15-4, IGHV2-10, IGHV2-26, IGHV2-70D, IGHV3-16, IGHV3-19, IGHV3-22, IGHV3-35, IGHV3-38, IGHV3-43D, IGHV3-47, IGHV3-52, IGHV3-64D, IGHV3-71, IGHV3-72, IGHV3-73, IGHV3-NL1, IGHV3-d, IGHV3-h, IGHV3/OR16-10, IGHV3/OR16-13, IGHV3/OR16-8, IGHV3/OR16-9, IGHV4-28, IGHV4-55, IGHV4/OR15-8, IGHV5-78, IGHV7-81, VH1-17P, VH1-67P, VH3-41P, VH3-60P, VH3-65P, VH7-27P; TRBV10-1, TRBV11-1, TRBV11-3, TRBV12-2, TRBV12-5, TRBV13, TRBV14, TRBV15, TRBV16, TRBV17, TRBV20/OR9-2, TRBV26, TRBV27, TRBV29/OR9-2, TRBV3-1, TRBV3-2, TRBV4-2, TRBV4-3, TRBV5-3, TRBV5-7, TRBV5-8, TRBV6-4, TRBV6-7, TRBV6-8, TRBV6-9, TRBV7-1, TRBV7-4, TRBV7-7. Most IGHV genes remaining after this filter had consistent, balanced prevalence across cohorts (FIGS. 29A and 29B).

Overlap with Database of Known SARS-COV-2 Binders

The Jul. 26, 2022 version of CoV-AbDab was downloaded, filtering to antibody sequences known to bind to SARS-COV-2 (including weak binders). Further, sequences from human patients or human antibody libraries were selected, and any IGHV genes that were never present in the dataset were removed, as these sequences would never be matched. The remaining SARS-COV-2 binders from CoV-AbDab with identical IGHV gene, IGHJ gene, and CDR-H3 lengths and at least 95% sequence identity were clustered. Several related sequences were combined and replaced by a consensus sequence.

Then overlapping sequences were found between the dataset and CoV-AbDab. First, in case of sequences in the dataset originating from different isotypes but sharing the same IGHV gene, IGHJ gene, and CDR-H3 sequence, the copy with highest predicted Covid-19 probability was kept, in order to assess the strength of a sequence's relationship to the disease. Then each sequence originating from a Covid-19 patient in the dataset (from any isotype) was assigned to the nearest CoV-AbDab cluster centroid, as long as they had the same IGHV gene, IGHJ gene, and CDR-H3 length and at least 85% sequence identity. Iterating over sequences in model-ranked order, starting with highest confidence sequences, the cumulative number of matches was counted to a cluster in the known binder database. An AUC score was also calculated using model rankings versus which BCR sequences matched the CoV-AbDab database. Finally, enrichment of these observed counts versus expected hits were calculated if sequences were ordered at random. The number of draws to sample a certain number of known binders without replacement from a pool of sequences follows the negative hypergeometric distribution. With N total sequences containing n<N known binders, a new known-binder is expected to be found with every

$$\frac{N + 1}{n + 1} = \frac{N - n}{n + 1} + 1$$

sequence draws.

### Repertoire Visualization

For each receptor, the lasso sequence model gives predicted class logits, which are proportional to the dot product of the embedded sequence vector and the model coefficients. In other words, this linear transformation applies the coefficients as weights on the input features, creating a sequences-by-classes matrix. To create a 2D visualization, UMAP was run on the per-disease-state logits for each sequence. Sequence labels were provided as supervision to the UMAP so they are less likely to be distorted in the layout.

A reference UMAP was created for each fold using a subset of training set sequences likely to be related to each disease state (or healthy). This subset of sequences was selected with the following filters:

First, to form the subset of sequences for a particular disease class, only sequences that originated from a patient with that disease were considered. Otherwise, the sequence could not plausibly be related to that disease. It would not make sense for a Covid-19 representative sequence to come from an HIV patient, for example.

Second, the lasso sequence model's prediction for this sequence must match the disease class, as well. After all, a reference layout was constructed of disease-specific sequences, so only include sequences the model has classified into the disease class should be included. Similarly, only sequences from the healthy class that originated from a healthy subject and are predicted to belong to that class were consider.

Third, sequences whose predictions were close calls were excluded. These borderline sequences were desired to be avoided in the construction of the reference map, especially because of the high label noise (described earlier). Therefore, potential sequences were filtered to those with predicted disease class probability at least 0.2 greater than probabilities predicted for any other class.

Finally, the remaining candidate sequences for each disease were sorted by their predicted probability of belonging to that disease state, and kept the top 20% to create a succinct pool of reference sequences for each class. The per-class logits for only these sequences were used to construct a UMAP.

Once the UMAP was constructed, held-out sequences were projected into the layout. First, therapeutic monoclonal antibodies were overlaid onto the 2D map. Their sequences were found via Thera-SabDab and annotated with IgBLAST. Supervised embeddings (per-class logits) were computed for each sequence using the sequence-level lasso model, and applied the trained UMAP transformation, producing 2D coordinates for each antibody.

Second, a held-out test patient's sequences was overlaid on the UMAP, applying the same process to a subset of the patient repertoire's sequences predicted to be disease-specific. The model and UMAP transformations belonging to the fold where the patient was in the held-out test set were used. The patient's repertoire was filtered to sequences whose predicted labels match the overall sample prediction by the ensemble metamodel, or sequences predicted to be Healthy/Background. As a result, the visualization included both the healthy and disease related components of this patient's B cell repertoire. Sequences to those with confident model predictions were further filtered: sequences having top predicted class probability at least 0.1 greater than the next highest class probability were chosen. All sequences remaining after these filtering steps were sorted by their predicted class probability. The top 20% of the sorted list across Healthy/Background and the overall sample predicted label class were kept.

1.-54. (canceled)

55. A method providing a computational surrogate assessment for an immune response based on B cell receptor sequences or T cell receptor sequences, comprising:
obtaining a collection of a biological sample from an individual comprising B cells or T cells;
extracting a genetic material from the biological sample, wherein the genetic material comprises DNA or RNA derived from the B cells or the T cells;
enriching and sequencing nucleic acids comprising sequences of B cell receptors or T cell receptors of the genetic material to yield a nucleic acid sequencing result of B cell receptors or T cell receptors of the individual; wherein the sequencing comprises high throughput sequencing; wherein each sequence of B cell receptors or T cell receptors comprises sequence for encoding a complementarity-determining region (CDR);
obtaining, using a computational processor, a receptor peptide sequence for each sequenced B cell receptor or T cell receptor from the nucleic acid sequencing result;
extracting, using the computational processor and a language model, a latent embedding of each receptor peptide sequence;
entering, using the computational processor, the latent embedding of each receptor peptide sequence in a trained classifier or regression model to predict a probability that a receptor peptide sequence is indicative of a presence of an immune response, wherein the trained classifier or regression has been trained utilizing extracted latent embeddings of receptor peptide sequences derived from a cohort of individuals having the autoimmune disorder; and
aggregating, using the computational processor, receptor peptide sequence probability predictions to yield a first sample-level probability prediction of the presence of the immune response.

56. The method of claim 55, wherein aggregating receptor peptide sequence probability predictions comprises:
computing a mean of the receptor peptide sequence probability predictions.

57. The method of claim 55 further comprising:
training, using the computational processor, the classifier or regression model by:
obtaining, using the computational processor, a B cell receptor or T cell receptor peptide sequences of two or more cohorts of individuals, wherein at least one cohort consists of individuals having an immune response; wherein each receptor peptide sequence comprises a complementarity-determining region (CDR) sequence of a B cell receptor or a T cell receptor;
extracting, using the computational processor and the language model, a latent embedding of each receptor peptide sequence;
labeling, using the computational processor, the latent embedding of each receptor peptide sequence with the cohort from which it was derived; and
entering, using the computational processor, the latent embedding of each receptor peptide sequence into the classifier or regression model to train the classifier or regression model to predict the probability that the receptor peptide sequence is indicative of the presence of an immune response.

58. The method of claim 57, wherein the at least one cohort consists of individuals having an immune response comprises a cohort having an autoimmune disorder.

59. The method of claim 57, wherein the B cell receptor or T cell receptor peptide sequences comprises receptor sequences labeled with known complementation to an antigen, wherein the antigen is associated with the immune response.

60. The method of claim 57 further comprising:
filtering, using the computational processor, the latent embeddings of receptor peptide sequences by:
clustering, using the computational processor, the latent embeddings of receptor peptide sequences; and
excluding, using the computational processor, a subset of the latent embeddings of receptor peptide sequences from being entered into the classifier or regression model for training; wherein the subset of the latent embeddings of receptor peptide sequences to be excluded are:
within a cluster having latent embeddings of receptor peptide sequences derived from of two or more cohorts; or
within a cluster having latent embeddings of receptor peptide sequences derived from only a minority of individuals within a cohort.

61. The method of claim 57 further comprising:
filtering, using the computational processor, the latent embeddings of receptor peptide sequences by:
constructing, using the computational processor, a nearest neighbors graph from latent embeddings of receptor peptide sequences; and
excluding, using the computational processor, a subset of the latent embeddings of receptor peptide sequences from being entered into the classifier or regression for training; wherein the subset of the latent embeddings of receptor peptide sequences to be excluded are:
within a graph neighborhood having latent embeddings derived from of two or more cohorts; or
within a graph neighborhood having latent embeddings of receptor peptide sequences derived from only a minority of individuals within a cohort.

62. The method of claim 55, wherein the language model embeds each peptide sequence into an internal, low-dimensional embedding.

63. The method of claim 55 further comprising:
optimizing a set of transformers of the language model using B cell receptor or T cell receptor peptide sequences to improve reconstruction accuracy of masked B cell receptor or T cell receptor peptide sequences within latent embeddings.

64. The method of claim 55 further comprising:
finetuning one or more parameters of the language model to improve classification of the presence of the immune response.

65. The method of claim 55 further comprising:
entering, using the computational processor, the HLA type as a covariate in the trained classifier or regression model.

66. The method of claim 55 further comprising
dimensionally reducing and projecting, using the computational processor, the extracted latent embeddings of receptor peptide sequences to visualize associations among the receptor peptide sequences.

67. The method of claim 55 further comprising:
generating, using the computational processor, a distribution of usage of V genes, J genes, or V-J gene pairs by B cells or T cells within a biological sample by:
tallying, using the computational processor and the B cell receptor or T cell receptor peptide sequences, for each B-cell clone or T-cell clone a usage of:
an immunoglobulin heavy chain variable (IGHV) gene, an immunoglobulin heavy chain joining (IGHJ) gene, or both IGHV and IGHJ; or
a T cell receptor beta chain variable (TRBV) gene, a T cell receptor beta chain joining (TRBJ) gene, or both TRBV and TRBJ to yield the distribution of usage of V genes, J genes, or V-J gene pairs;
entering, using the computational processor, the distribution of usage of V genes, J genes, or V-J gene pairs into a second trained classifier or regression model to predict a second sample-level probability prediction of the presence of the immune response, wherein the second trained classifier or regression model is trained utilizing distributions of usage of V genes, J genes, or V-J gene pairs derived from the cohort of individuals having the immune response and distributions of usage of V genes, J genes, or V-J gene pairs derived from a cohort of individuals not having the immune response; and
entering, using the computational processor, the first sample-level probability prediction and the second sample-level probability prediction into a composite trained classifier or regression model to predict a composite sample-level probability prediction, wherein the composite trained classifier or regression model is trained utilizing:
first sample-level probability predictions and second sample-level probability predictions derived from the cohort of individuals having the immune response; and
first sample-level probability predictions and second sample-level probability predictions derived from a cohort of individuals not having the immune response.

68. The method of claim 55 further comprising:
grouping, using the computational processor, the B cell receptor or T cell receptor peptide sequences into clusters;
assigning, using the computational processor, each B cell receptor or T cell receptor peptide sequence to a cluster to yield a set of cluster memberships;
entering, using the computational processor, the set of cluster memberships into a second trained classifier or regression model to predict a second sample-level probability prediction of the presence of the immune response, wherein the second trained classifier or regression model is trained utilizing sets of cluster memberships derived from the cohort of individuals having the immune response and sets of cluster memberships derived from a cohort of individuals not having the immune response; and
entering, using the computational processor, the first sample-level probability prediction and the second sample-level probability prediction into a composite trained classifier or regression model to predict a composite sample-level probability prediction, wherein the composite trained classifier or regression model is trained utilizing:
first sample-level probability predictions and second sample-level probability predictions from the cohort of individuals having the immune response, and
first sample-level probability predictions and second sample-level probability predictions derived a cohort of individuals not having the immune response.

69. The method of claim 55 further comprising:
generating, using the computational processor, a distribution of usage of V genes, J genes, or V-J gene pairs by B cells or T cells within biological sample by:
tallying, using the computational processor and the B cell receptor or T cell receptor peptide sequences, for each B-cell clone or T-cell clone a usage of:
an immunoglobulin heavy chain variable (IGHV) gene, an immunoglobulin heavy chain joining (IGHJ) gene, or both IGHV and IGHJ; or
a T cell receptor beta chain variable (TRBV) gene, a T cell receptor beta chain joining (TRBJ) gene, or both TRBV and TRBJ to yield the distribution of usage of V genes, J genes, or V-J gene pairs;
entering, using the computational processor, the distribution of usage of V genes, J genes, or V-J gene pairs into a second trained classifier or regression model to predict a second sample-level probability prediction of the presence of the immune response, wherein the second trained classifier or regression model is trained utilizing distributions of usage of V genes, J genes, or V-J gene pairs derived from the cohort of individuals having the immune response and distributions of usage of V genes, J genes, or V-J gene pairs derived from a cohort of individuals not having the immune response;
grouping, using the computational processor, the B cell receptor or T cell receptor peptide sequences into clusters;
assigning, using the computational processor, each B cell receptor or T cell receptor peptide sequence to a cluster to yield a set of cluster memberships;
entering, using the computational processor, the set of cluster memberships into a third trained classifier or regression model to predict a third sample-level probability prediction of the presence of the immune response, wherein the third trained classifier or regression model is trained utilizing sets of cluster memberships derived from the cohort of individuals having the immune response and sets of cluster memberships derived a cohort of individuals not having the immune response; and
entering, using the computational processor, the first sample-level probability prediction, the second sample-level probability prediction, and the third sample-level probability prediction into a composite trained classifier or regression model to predict a composite sample-level probability prediction, wherein the composite trained classifier or regression model is trained utilizing:
first sample-level probability predictions, second sample-level probability predictions, and third sample-level probability predictions from the cohort of individuals having the immune response; and
first sample-level probability predictions, second sample-level probability predictions, and third sample-level probability predictions derived a cohort of individuals not having the immune response.

70. The method of claim 55, wherein a B cell sample-level probability prediction of a presence of the immune response is determined utilizing B cell receptor peptide sequences and wherein a T cell sample-level probability prediction of a presence of the immune response is determined utilizing T cell receptor peptide sequences; wherein the method further comprises:
entering, using the computational processor, the B cell sample-level probability prediction and the T cell sample-level probability prediction into a composite trained classifier or regression model to predict a composite sample-level probability prediction, wherein the composite trained classifier or regression model is trained utilizing:
B cell sample-level probability predictions and T cell sample-level probability predictions derived from the cohort of individuals having the immune response; and
B cell sample-level probability predictions and T cell sample-level probability predictions derived from a cohort of individuals not having the immune response.

71. The method of claim 55, wherein the immune response indicates presence of an autoimmune disorder, the method further comprising:
indicating, using the computational processor, the individual has the autoimmune disorder based on the first sample-level probability prediction of a presence of the autoimmune disorder.

72. The method of claim 71 further comprising:
administering an immunity suppression treatment to the individual to treat the autoimmune disorder.

73. The method of claim 71, wherein the first sample-level probability prediction further yields a sample-level probability prediction of whether the individual is experiencing an active flare; and
indicating, using the computational processor, the individual is experiencing the active autoimmune disorder flare based on the sample-level probability prediction of whether the individual is experiencing the active flare.

74. The method of claim 71, wherein the first sample-level probability prediction further yields a sample-level probability prediction of a presence of a subtype of an autoimmune disorder; and
indicating, using the computational processor, the individual as having the subtype of the autoimmune disorder based on the subtype sample-level probability prediction of the presence of the subtype of the autoimmune disorder and that an immunity suppression treatment to be administered is based on the immunity suppression treatment having efficacy on the subtype of autoimmune disorder.

75. The method of claim 71, wherein the first sample-level probability prediction further yields a sample-level probability prediction of severity of the autoimmune disorder; and
indicating, using the computational processor, the individual as having a severe autoimmune disorder based on the severity sample-level probability prediction and that an immunity suppression treatment to be administered is based on the immunity suppression treatment having efficacy on the severity of the autoimmune disorder.

76. The method of claim 55, wherein the immune response indicates presence of an autoimmune disorder, the method further comprising:
administering an immunity suppression treatment to the individual; and
monitoring the immunity suppression treatment by intermittently determining, using the computational processor, a subsequent sample-level probability prediction of the presence of the autoimmune disorder, wherein the subsequent sample-level probability prediction is determined from a subsequent collection of a biological sample that is collected after the immunity suppression treatment has begun.

77. The method of claim 76 further comprising:
indicating, using the computational processor, that the subsequent sample-level probability prediction of a presence of the autoimmune disorder predicts that the autoimmune disorder has waned; and
altering the immunity suppression treatment based on the waning of the autoimmune disorder.

78. The method of claim 55, wherein the immune response is a response to systemic lupus erythematosus.

79. The method of claim 55, wherein the B cell receptor or T cell receptor peptide sequences comprises 10,000 or more peptide sequences.
